SEQUENCE LISTING (23 sequences)

SEQ ID NO: 1 - DNA, 20 bp, Artificial
  Amplification primer
ggtcaacatctggctttccg

SEQ ID NO: 2 - DNA, 22 bp, Artificial
  Amplification primer
caccagtctgtgagtggaccaa

SEQ ID NO: 3 - DNA, 20 bp, Artificial
  Amplification primer
actgaagcactacgggcctg

SEQ ID NO: 4 - DNA, 19 bp, Artificial
  Amplification primer
agccgttggtgtctttgcc

SEQ ID NO: 5 - DNA, 20 bp, Artificial
  Amplification primer
ggtcaaatttaccctggcca

SEQ ID NO: 6 - DNA, 22 bp, Artificial
  Amplification primer
tctcatcaagcgtcagcagttc

SEQ ID NO: 7 - DNA, 19 bp, Artificial
  Amplification primer
tggaacggtgaaggtgaca

SEQ ID NO: 8 - DNA, 19 bp, Artificial
  Amplification primer
ggcaagggacttcctgtaa

SEQ ID NO: 9 - DNA, 20 bp, Artificial
  Amplification primer
cgtcatgggtgtgaaccatg

SEQ ID NO: 10 - DNA, 21 bp, Artificial
  Amplification primer
gctaagcagttggtggtgcag

SEQ ID NO: 11 - DNA, 21 bp, Artificial
  Amplification primer
gtcgctggtcagttcgtgatt

SEQ ID NO: 12 - DNA, 23 bp, Artificial
  Amplification primer
agcagttggctgttgtacctctc

SEQ ID NO: 13 - DNA, 20 bp, Artificial
  Amplification primer
aatgcatgctaccaaaagag

SEQ ID NO: 14 - DNA, 20 bp, Artificial
  Amplification primer
aatcatttgattcccaggac

SEQ ID NO: 15 - DNA, 15 bp, Artificial
  Sequencing primer
ggcagagccttgccc

SEQ ID NO: 16 - DNA, 21 bp, Homo sapiens
agtgtcctagttcattttcac

SEQ ID NO: 17 - DNA, 20 bp, Homo sapiens
ttacaatactgtctggtgga

SEQ ID NO: 18 - DNA, 21 bp, Homo sapiens
agtgtcctagttcattttcac

SEQ ID NO: 19 - DNA, 20 bp, Homo sapiens
ttacaatactgtccggtgga

SEQ ID NO: 20 - DNA, 21 bp, Homo sapiens
agtgtccttgttcattttcac

SEQ ID NO: 21 - DNA, 20 bp, Homo sapiens
ttacaatactgtccggtgga

SEQ ID NO: 22 - DNA, 21 bp, Homo sapiens
agtgtccttgttcattttcac

SEQ ID NO: 23 - DNA, 20 bp, Homo sapiens
ttacaatactgtctggtgga

(19) United States
(12) Patent Application Publication
(10) Pub. No.: US 2005/0177881 A1
(43) Pub. Date: Aug. 11, 2005

(54) Methods of identifying genetic risk for and evaluating treatment of alzheimer's disease by determining single nucleotide polymorphisms

(76) Inventors: Andreas Papassotiropoulos, Zurich (CH); Johannes R. Streffer, Zurich (CH); Christoph Hock, Zurich (CH); Roger Nitsch, Zollikon (CH)

Correspondence Address: JACOBSON HOLMAN PLLC, 400 SEVENTH STREET N.W., SUITE 600, WASHINGTON, DC, 20004, US

(21) Appl. No.: 10/497,590

(22) PCT Filed: Dec. 3, 2002
(86) PCT No.: PCT/EP02/13632

Related U.S. Application Data
(60) Provisional application No. 60/334,966, filed on Dec. 4, 2001.

(30) Foreign Application Priority Data
Dec. 4, 2001 (EP) 01128827.1
Jan. 23, 2002 (EP) 02001577.2

(51) Int. Cl. A01K 67/027; C12Q 1/68; A61K 49/00

(52) U.S. Cl. 800/12; 435/6; 424/9.2

(57) ABSTRACT

Based on the unexpected identification of single nucleotide polymorphisms in the LIPA gene as novel genetic risk factors that link cholesterol metabolism to Alzheimer's disease, the present invention provides a method of diagnosing or prognosticating Alzheimer's disease, or determining the propensity or predisposition of a subject to develop Alzheimer's disease. The method comprises detecting the presence or absence of a variation in the LIPA gene which encodes the enzyme acid cholesteryl ester hydrolase. Furthermore, the invention provides methods of diagnosing Alzheimer's disease by using the CH25H gene and/or the LIPA gene and their corresponding gene products.

The present invention relates to methods of diagnosing, prognosticating, or determining the predisposition of a subject to develop a neurodegenerative disease. Furthermore, methods of therapy control and screening for modulating agents of neurodegenerative diseases are provided.

Neurodegenerative diseases, in particular Alzheimer's disease (AD), have a strongly debilitating impact on a patient's life. Furthermore, these diseases constitute an enormous health, social, and economic burden. AD is the most common neurodegenerative disease, accounting for about 70% of all dementia cases, and it is probably the most devastating age-related neurodegenerative condition, affecting about 10% of the population over 65 years of age and up to 45% over age 85 (for a recent review see Vickers et al., Progress in Neurobiology 2000, 60:139-165). Presently, this amounts to an estimated 12 million cases in the US, Europe, and Japan. This situation will inevitably worsen with the demographic increase in the number of old people (“aging of the baby boomers”) in developed countries. The neuropathological hallmarks that occur in the brain of individuals suffering from Alzheimer's disease are senile plaques, composed of amyloid-β protein, and profound cytoskeletal changes coinciding with the appearance of abnormal filamentous structures and the formation of neurofibrillary tangles. AD is a progressive disease that is associated with early deficits in memory formation and ultimately leads to the complete erosion of higher cognitive function Alzheimer's disease is genetically complex. The risk for the development of AD is determined by variations of genes involved in major pathophysiological pathways of this disorder. A considerable part of this risk is attributed to the inheritance of the e4 allele of the apolipoprotein E gene (APOE*4). However, several additional genes and genetic interactions add to the overall genetically determined susceptibility for the development of AD.

Genes coding for proteins involved in central disease-related pathways are of particular interest in the genetics of AD. The overproduction and aggregation of the β-amyloid peptide (Aβ) in the hippocampus and the medial lobe (MTL) is a crucial step in the pathogenesis of AD. Thus, genes implicated in mechanisms leading to Aβ accumulation are promising candidates in the search for susceptibility genes of AD.

Brain deposition of β-amyloid peptide (Aβ) is a crucial step in the pathogenesis of AD (Hardy J A, et al., Science, 256:184-5, 1992). It can cause the formation of neurofibrillary tangles within neurons (Götz J, et al., Science, 293:1491-5, 2001; Lewis J, et al., Science, 293:1487-91, 2001). The concentration of the amyloid peptide Aβ42 may be used as a surrogate, quantitative trait to identify genetic loci for AD (Ertekin-Taner N, et al., Science, 290:2303-4, 2000). Thus, genes implicated in the regulation of Aβ formation and its degradation are candidate susceptibility genes for AD. Recent observations link brain levels of cholesterol to the regulation of the endoproteolytic processing of APP, and to Aβ production (Simons M, et al., Neurology, 57:1089-93, 2001; Puglielli L, et al., Nature Cell Biology, 3:905-912, 2001). Cholesterol depletion inhibits the production of Aβ in vitro (Simons M, et al., Proc Natl Acad Sci USA, 95:6460-4, 1998), and such cholesterol-lowering drugs as statins reduce the levels of Aβ in vitro and in vivo (Fassbender K, et al., Proc Natl Acad Sci USA, 98:5856-61, 2001). Moreover, clinical observations suggest that statins reduce the risk for dementia and AD (Jick H, et al., Lancet, 356:1627-31, 2000; Wolozin B, et al., Arch Neurol, 57:1439-43, 2000).

One key enzyme involved in cholesterol metabolism is lysosomal acid lipase (LIPA), also known as acid cholesteryl ester hydrolase. The human gene encoding LIPA is located on chromosome 10. More specifically, the LIPA gene maps to the cytogenetic band 10q23.2-q23.3 (Anderson et al, Genomics, 15:245-247, 1993). The GenBank accession number for LIPA is NM000235. The investigation of the genomic organization of the LIPA gene revealed the existence of 10 exons (Aslanidis et al., Genomics 20:329-31, 1994). Lysosomal acid lipase, subcellularly located mainly in the lysosome compartment, exerts a critical function in the intracellular hydrolysis of cholesteryl esters and triglycerides which have been internalized by receptor-mediated endocytosis of lipoprotein particles. Two genetic disorders, the severe early-onset Wolman disease and the milder late-onset cholesteryl ester storage disease (CESD), are caused by mutations in different parts of the LIPA gene (Anderson et al., Proc. Natl. Acad. Sci., 91:2718-2722, 1994). Both of the above disorders are associated with reduced activity of lysosomal acid lipase (Aslanidis et al., Genomics, 33:85-93, 1996). A characteristic histopathological feature of both of these chronic liver diseases is the excessive lysosomal storage of triglycerides and cholesterol esters and adrenal calcification. However, lipid inclusions are not limited to cells of the hepatic system but have also been reported in cells of the central and peripheral nervous system (Byrd and Powers, Acta Neuropathol, 45:37-42, 1979).

The CH25H gene codes for cholesterol 25-hydroxylase, another key enzyme of cholesterol metabolism. The CH25H gene (GenBank accession number: NM 003956) is located in very close vicinity of the genomic locus of LIPA (i.e. approximately 6000 bp further downstream of the 3′-end of the LIPA gene). Cholesterol 25-hydroxylase catalyzes the hydroxylation of hydrophobic substrates, thereby converting cholesterol to an oxysterol (Russell and Lund, WO0023596; Lund et al., J Biol Chem, 273:34316-34327, 1998). The level of oxysterols, in particular of 25-hydroxycholesterol, in a cell suppresses the proteolytic processing of the sterol response element binding protein (SREBP), thereby negatively regulating sterol synthesis (Russell, Biochim Biophys Acta, 1529:126-135, 2000). The LIPA gene and the CH25H gene are situated very close together, so that both genes may overlap in respect to their regulatory regions. Accordingly, LIPA and CH25H, within the context of the chromosomal DNA segment comprising both genes, may also share certain regulatory elements that are critical for their expression. Due to this very close proximity of CH25H to LIPA, variations, such as single nucleotide polymorphisms, in the nucleotide sequence of one of the two genes may have an influence or an impact on the activity and function of the other gene, or both genes. Furthermore, it is possible that an observed association between a variation in the nucleotide sequence of the LIPA gene and Alzheimer's disease may be attributable to linkage disequilibrium (LD) with a distinct locus within the LIPA gene or in the close vicinity of the LIPA gene, such as in the CH25H gene. Likewise, it is possible that an observed association between a variation in the nucleotide sequence of the CH25H gene and Alzheimer's disease may be attributable to linkage disequilibrium with a distinct locus within the CH25H gene or in the close vicinity of the CH25H gene, such as in the LIPA gene.

Yet one further important enzyme in cholesterol metabolism is cholesterol 24-hydroxylase. The enzyme plays an important role in cholesterol removal from the brain (Lund E G, et al., Proc Natl Acad Sci USA, 96:7238-43, 1999) by catalyzing the conversion of cholesterol to 24S-hydroxycholesterol (24-OH-Chol), which readily crosses the blood-brain-barrier (Lutjohann D, et al., Proc Natl Acad Sci USA, 93:9799-804, 1996). Hydroxylation is therefore the rate limiting step in cholesterol removal from brain (Bjorkhem I, et al., J Biol Chem, 272:30178-84, 1997; ibid, J Lipid Res, 39:1594-600, 1998). The gene encoding cholesterol 24-hydroxylase, CYP46, is a member of the cytochrome P450 subfamily; it maps to chromosome 14q32.1 (GenBank accession numbers: NM006668; XM007242; AF094480). In humans, CYP46 is expressed predominantly in the brain, with mRNA mainly found in the gray matter. In situ hybridizations of mouse brains showed abundant mRNA in neurons of the cerebral cortex, hippocampus, dentate gyrus, and the thalamus.

It was an objective of the present invention to provide methods of diagnosing or prognosticating Alzheimer's disease. A further objective of the present invention was to provide methods of monitoring the progression of this disease and of evaluating a treatment for it. This objective was based on the identification of a single nucleotide polymorphism in the LIPA gene as a novel genetic risk factor that links cholesterol metabolism to Alzheimer's disease. The objective of the present invention has been solved by the methods and kits according to the features of the independent claims. Further preferred embodiments of the present invention are defined in the sub-claims thereto.

The singular forms “a”, “an”, and “the” as used herein and in the claims include plural reference unless the context dictates otherwise. For example, “a cell” means as well a plurality of cells, and so forth. The term “and/or” as used in the present specification and in the claims implies that the phrases before and after this term are to be considered either as alternatives or in combination. For instance, the wording “determination of a level and/or an activity” means that either only a level, or only an activity, or both a level and an activity are determined. The term “level” as used herein is meant to comprise a gage of, or a measure of the amount of, or a concentration of a transcription product, for instance an mRNA, or a translation product, for instance a protein or polypeptide. The term “activity” as used herein shall be understood as a measure for the ability of a transcription product or a translation product to produce a biological effect or a measure for a level of biologically active molecules. The term “activity” also refers to enzymatic activity. The terms “level” and/or “activity” as used herein further refer to gene expression levels or gene activity. Gene expression can be defined as the utilization of the information contained in a gene by transcription and translation leading to the production of a gene product. “Dysregulation” shall mean an upregulation or downregulation of gene expression. A gene product comprises either RNA or protein and is the result of expression of such a gene. The amount of a gene product can be used to measure how active a gene is. The term “gene” as used in the present specification and in the claims comprises both coding regions (exons) as well as non-coding regions (e.g. non-coding regulatory elements such as promoters or enhancers, introns, leader and trailer sequences). The term “ORF” is an acronym for “open reading frame” and refers to a nucleic acid sequence that does not possess a stop codon in at least one reading frame and therefore can potentially be translated into a sequence of amino acids. “Regulatory elements” shall comprise inducible and non-inducible promoters, enhancers, operators, and other elements that drive and regulate gene expression. The term “fragment” as used herein is meant to comprise e.g. an alternatively spliced, or truncated, or otherwise cleaved transcription product or translation product. The term “derivative” as used herein refers to a mutant, or an RNA-edited, or a chemically modified, or otherwise altered transcription product, or to a mutant, or chemically modified, or otherwise altered translation product. For instance, a “derivative” may be generated by processes such as altered phosphorylation, or glycosylation, or, acetylation, or lipidation, or by altered signal peptide cleavage or other types of maturation cleavage. These processes may occur post-translationally. The term “modulator” as used in the present invention and in the claims refers to a molecule capable of changing or altering the level and/or the activity of a gene, or a transcription product of a gene, or a translation product of a gene. Preferably, a “modulator” is capable of changing or altering the biological activity of a transcription product or a translation product of a gene. Said modulation, for instance, may be an increase or a decrease in enzyme activity, a change in binding characteristics, or any other change or alteration in the biological, functional, or immunological properties of said translation product of a gene. The terms “modulator”, “agent”, “reagent”, or “compound” refer to any substance, chemical, composition or extract that have a positive or negative biological effect on a cell, tissue, body fluid, or within the context of any biological system, or any assay system examined. They can be agonists, antagonists, partial agonists or inverse agonists of a target. Such agents, reagents, or compounds may be nucleic acids, natural or synthetic peptides or protein complexes, or fusion proteins. They may also be antibodies, organic or anorganic molecules or compositions, small molecules, drugs and any combinations of any of said agents above. They may be used for testing, for diagnostic or for therapeutic purposes. The terms “oligonucleotide primer” or “primer” refer to short nucleic acid sequences which can anneal to a given target polynucleotide by hybridization of the complementary base pairs and can be extended by a polymerase. They may be chosen to be specific to a particular sequence or they may be randomly selected, e.g. they will prime all possible sequences in a mix. The length of primers used herein may vary from 10 nucleotides to 80 nucleotides. “Probes” are short nucleic acid sequences of the nucleic acid sequences described and disclosed herein or sequences complementary therewith. They may comprise full length sequences, or fragments, derivatives, isoforms, or variants of a given sequence. The identification of hybridization complexes between a “probe” and an assayed sample allows the detection of the presence of other similar sequences within that sample. As used herein, “homolog or homology” is a term used in the art to describe the relatedness of a nucleotide or peptide sequence to another nucleotide or peptide sequence, which is determined by the degree of identity and/or similarity between said sequences compared. The term “variant” as used herein refers to any polypeptide or protein, in reference to polypeptides and proteins disclosed in the present invention, in which one or more amino acids are added and/or substituted and/or deleted and/or inserted at the N-terminus, and/or the C-terminus, and/or within the native amino acid sequences of the native polypeptides or proteins of the present invention. Furthermore, the term “variant” shall include any shorter or longer version of a polypeptide or protein. “Variants” shall also comprise a sequence that has at least about 80% sequence identity, more preferably at-least about 90% sequence identity, and most preferably at least about 95% sequence identity with the amino acid sequences of a polypeptide or protein. Such “variants” include, for example, polypeptides or proteins with conservative amino acid substitutions in highly conservative regions. “Proteins and polypeptides” of the present invention include variants, fragments and chemical derivatives of a protein or polypeptide as disclosed in the present invention. They can include proteins and polypeptides which can be isolated from nature or be produced by recombinant and/or synthetic means. Native proteins or polypeptides refer to naturally-occurring truncated or secreted forms, naturally occurring variant forms (e.g. splice-variants) and naturally occurring allelic variants. The term “isolated” as used herein is considered to refer to molecules that are removed from their natural environment, i.e. isolated from a cell or from a living organism in which they normally occur, and that are separated or essentially purified from the coexisting components with which they are found to be associated in nature. This notion further means that the sequences encoding such molecules can be linked by the hand of man to polynucleotides, to which they are not linked in their natural state, and that such molecules can be produced by recombinant and/or synthetic means. Even if for said purposes those sequences may be introduced into living or non-living organisms by methods known to those skilled in the art, and even if those sequences are still present in said organisms, they are still considered to be isolated. In the present invention, the terms “risk”, “susceptibility”, and “predisposition” are tantamount and are used with respect to the probability of developing a neurodegenerative disease, preferably Alzheimer's disease.

The term “polymorphism” refers to the existence of more than one form of a gene or portion of a gene. It refers to a genetic variation in a nucleotide sequence at a given nucleotide position in the genome, within a given population, and a frequency usually exceeding 1%. Regions harboring polymorphisms may be a given gene region, coding or non-coding portions of the gene, or even intergenic regions, and are designated as “polymorphic regions”. They may cause differences in the nucleotide sequences as well as in the polypeptide sequences, in protein modifications, gene and protein expression processes and DNA replication. The term “single nucleotide polymorphism (SNP)” refers to a polymorphic variation in a nucleotide sequence at a given single nucleotide position in the genome. Single nucleotide polymorphisms may include any single base changes such as a deletion, insertion, or a base exchange. A single nucleotide polymorphism may cause a change in the encoded polypeptide sequence as well. A particular SNP may be indicative for a disease state, a specific feature, or for the risk of developing a disease. The term “allele” or “allelic variant” refers to one of several alternative forms of a gene, or a portion thereof, typically having particular features which result in a particular phenotype. The term “allele” includes any inherited variation in the DNA sequence of a gene located at a given position in the genome. An individual or a subject is “homozygous” when two alleles of a given gene of a diploid organism are identical in respect to a given variation or polymorphism. The term “haplotype” refers to the polymorphisms located on a single DNA strand and to a series of alleles at several closely linked gene loci on a single chromosome. The term “linkage disequilibrium” refers to alleles which are nonrandomly associated at closely linked gene loci and operates over distances less than 1 cM.

The term “AD” shall mean Alzheimer's disease. “AD-type neuropathology” as used herein refers to neuropathological, neurophysiological, histopathological and clinical hallmarks as described in the instant invention and as commonly known from state-of-the-art literature (see: Iqbal, Swaab, Winblad and Wisniewski, Alzheimer's Disease and Related Disorders (Etiology, Pathogenesis and Therapeutics), Wiley & Sons, New York, Weinheim, Toronto, 1999; Scinto and Daffner, Early Diagnosis of Alzheimer's Disease, Humana Press, Totowa, N.J., 2000; Mayeux and Christen, Epidemiology of Alzheimer's Disease: From Gene to Prevention, Springer Press, Berlin, Heidelberg, New York, 1999; Younkin, Tanzi and Christen, Presenilins and Alzheimer's Disease, Springer Press, Berlin, Heidelberg, New York, 1998).

Neurodegenerative diseases or disorders according to the present invention comprise Alzheimer's disease, Parkinson's disease, Huntington's disease, amyotrophic lateral sclerosis, Pick's disease, fronto-temporal dementia, progressive nuclear palsy, corticobasal degeneration, cerebro-vascular dementia, multiple system atrophy, argyrophilic grain dementia and other tauopathies, and mild-cognitive impairment. Further conditions involving neurodegenerative processes are, for instance, ischemic stroke, age-related macular degeneration, narcolepsy, motor neuron diseases, prion diseases, traumatic nerve injury and repair, and multiple sclerosis.

In one aspect, the invention features a method for diagnosing or prognosticating Alzheimer's disease in a subject, or determininig the propensity or predisposition of a subject to develop Alzheimer's disease. The method comprises detecting in a sample obtained from said subject the presence or absence of a variation in the LIPA gene, wherein the presence of a variation in the LIPA gene in said subject indicates a diagnosis or prognosis of Alzheimer's disease, or an increased propensity or predisposition of developing Alzheimer's disease as compared to a subject who does not carry a variation in said gene. The LIPA gene codes for the enzyme lysosomal acid lipase, also called acid cholesteryl ester hydrolase. The GenBank accession number for the LIPA gene is NM000235. The terms “propensity” or “predisposition” as employed herein are used interchangeably with reference to developing Alzheimer's disease and are tantamount to the terms “susceptibility” or “risk”. A variation in the LIPA gene can be understood as any alteration in the naturally occuring nucleic acid sequence of the LIPA gene, i.e. any alteration from the wildtype. The variation may be present in one copy or in both copies of the LIPA gene, in other words, the subject may be heterozygous or homozygous for said variation.

In a preferred embodiment, the variation in the LIPA gene is a single nucleotide polymorphism in the 3′- untranslated region of the gene (single nucleotide polymorphism identification number: rs13500). In a further preferred embodiment, the variation is a C to T transition.

In another preferred embodiment, the variation in the LIPA gene is a single nucleotide polymorphism in the 3′- untranslated region of the gene (single nucleotide polymorphism identification number: rs1131706). Preferably, said variation is an A to T transversion.

In a particular embodiment of the instant invention, a diagnosis or prognosis of Alzheimer's disease, or an increased propensity or predisposition to develop Alzheimer's disease in a subject, is indicated by the presence of both variations, the T-variant of SNP rs13500 and the T-variant of SNP rs1131706, in comparison to a subject who does not carry both of said variations, i.e. a subject who carries the C-variant of SNP rs13500 and the A-variant of SNP rs1131706.

The method, according to the present invention, may be particularly useful for the identification of individuals that are at risk of developing Alzheimer's disease. Consequently, the method, according to the present invention, may serve as a means for targeting identified individuals for early preventive measures or therapeutic intervention prior to disease onset, before irreversible damage in the course of the disease has been inflicted.

Determining the presence or absence of a polymorphism or variation in the LIPA gene may comprise determining a partial nucleotide sequence of the DNA from said subject, said partial nucleotide sequence indicating the presence or absence of said polymorphism or variation. It may further be preferred to perform a polymerase chain reaction with the DNA from said subject to determine the presence or absence of said polymorphism or variation. Such techniques are known to those skilled in the art (see Lewin B, Genes V, Oxford University Press, 1994).

In a preferred embodiment of the invention, the method further comprises detecting in a sample from said subject the presence of an apolipoprotein E4 allele, wherein the presence of one or both of the variations in the LIPA gene and the presence of an apolipoprotein E4 allele in said subject indicates a diagnosis or prognosis of Alzheimer's disease, or a further increased propensity or predisposition to develop Alzheimer's disease as compared to a subject who carries either only one or both of said variations in the LIPA gene or only an apolipoprotein E4 allele, or neither one or both of said variations in the LIPA gene and an apolipoprotein E4 allele. The method of this embodiment reflects the surprising finding of an unexpected synergistic interaction between the genes coding for lysosomal acid lipase (LIPA) and/or cholesterol 25-hydroxylase (CH25H) and apolipoprotein E4.

In another preferred embodiment of the invention, the method further comprises detecting in a sample from said subject the presence of a variation in the CYP46 gene, wherein the presence of one or both of the variations in the LIPA gene and the presence of a variation in the CYP46 gene in said subject indicates a diagnosis or prognosis of Alzheimer's disease, or a further increased propensity or predisposition to develop Alzheimer's disease as compared to a subject who carries either only one or both of said variations in the LIPA gene or only said variation in the CYP46, or neither said variations in the LIPA gene and said variation in the CYP46 gene. It is preferred that said variation in the CYP46 gene is a single nucleotide polymorphism at a position 151 bp 5′ of exon 3 (single nucleotide polymorphism indentification number: rs754203). It is further preferred that said variation is a C to T transition and that the variation is present in both alleles of said subject, that is, the subject is homozygous in respect to said variation (i.e. the CYP46*TT genotype). The method of this embodiment reflects the surprising finding of an unexpected synergistic interaction between the genes coding for lysosomal acid lipase (LIPA) and/or cholesterol 25-hydroxylase (CH25H) and cholesterol 24-hydroxylase (CYP46).

In a preferred embodiment of the invention, the sample taken from a subject for analysis comprises DNA obtained from body fluids, tissues, or any suitable cells readily available. Preferably, the sample is a blood sample. However, the sample may also consist of body fluids such as saliva, urine, serum plasma, nasal mucosa, or cerebrospinal fluid.

In a further aspect, the invention features a method for diagnosing or prognosticating a neurodegenerative disease in a subject, or determining the propensity or predisposition of a subject to develop said disease, comprising: determining a level, or an activity, or both said level and said activity, of at least one substance which is selected from the group consisting of a transcription product of the LIPA gene, or a translation product of the LIPA gene in a sample from said subject; and comparing said level, or said activity, or both said level and said activity, of at least one of said substances to a reference value representing a known disease or health status, thereby diagnosing or prognosticating said neurodegenerative disease in said subject, or determining the propensity or predisposition of said subject to develop said neurodegenerative disease. Due to the very close proximity of the CH25H gene to the LIPA gene, variations, such as single nucleotide polymorphisms, in the nucleotide sequence of one of the two genes may have an influence or an impact on the activity and function of the other gene, or both genes. Furthermore, it is possible that an observed association between a variation in the nucleotide sequence of the LIPA gene and Alzheimer's disease may be attributable to linkage disequilibrium (LD) with a distinct locus within the LIPA gene or in the close vicinity of the LIPA gene, such as in the CH25H gene. Therefore, it may be desirable to further determine a level, or an activity, or both said level and said activity, of at least one substance which is selected from the group consisting of a transcription product of the CH25H gene, or a translation product of the CH25H gene in a sample from said subject.

The invention also relates to the construction and the use of primers and probes which are unique to the nucleic acid sequences, or fragments or variants thereof, as disclosed in the present invention. The oligonucleotide primers and/or probes can be labeled specifically with fluorescent, bioluminescent, magnetic, or radioactive substances. The invention further relates to the detection and the production of said nucleic acid sequences, or fragments and variants thereof, using said specific oligonucleotide primers in appropriate combinations. PCR-analysis, a method well known to those skilled in the art, can be performed with said primer combinations to amplify said gene specific nucleic acid sequences from a sample containing nucleic acids. Such sample may be derived either from healthy or diseased subjects. Whether an amplification results in a specific nucleic acid product or not, and whether a fragment of different length can be obtained or not, may be indicative for a neurodegenerative disease, in particular Alzheimer's disease. Thus, the invention provides nucleic acid sequences, oligonucleotide primers, and probes of at least 10 bases in length up to the entire coding and gene sequences, useful for the detection of gene mutations and single nucleotide polymorphisms in a given sample comprising nucleic acid sequences to be examined, which may be associated with neurodegenerative diseases, in particular Alzheimers disease. This feature has utility for developing rapid DNA-based diagnostic tests, preferably also in the format of a kit.

In another aspect, the present invention provides a method of monitoring the progression of a neurodegenerative disease in a subject, comprising: determining a level, or an activity, or both said level and said activity, of at least one substance which is selected from the group consisting of a transcription product of the LIPA gene and/or the CH25H gene, or a translation product of the LIPA gene and/or the CH25H gene, in a sample from said subject; and comparing said level, or said activity, or both said level and said activity, of at least one of said substances to a reference value representing a known disease or health status, thereby monitoring the progression of said neurodegenerative disease in said subject.

In a further aspect, the present invention provides a method of evaluating a treatment for a neurodegenerative disease, comprising determining a level, or an activity, or both said level and said activity, of at least one substance which is selected from the group consisting of a transcription product of the LIPA gene and/or the CH25H gene, or a translation product of the LIPA gene and/or the CH25H gene, in a sample obtained from a subject being treated for said disease; and comparing said level, or said activity, or both said level and said activity, of at least one of said substances to a reference value representing a known disease or health status, thereby evaluating said treatment for said neurodegenerative disease.

In a further preferred embodiment of the herein claimed methods, kits, recombinant animals, molecules, assays, and uses of the instant invention, said neurodegenerative disease or disorder is Alzheimer's disease, and said subjects suffer from Alzheimer's disease.

In a preferred embodiment of the invention, the sample to be analyzed is taken from a body fluid, preferably cerebrospinal fluid, saliva, urine, mucus, nasal mucosa, blood or serum plasma, or a tissue, or cells like skin fibroblasts. Most preferably, the sample is taken from cerebrospinal fluid or blood. Preferably, the methods of diagnosis, prognosis, monitoring the progression or evaluating a treatment for a neurodegenerative disease, according to the instant invention, can be practiced ex corpore, and such methods preferably relate to samples, for instance body fluids or cells, that have been removed, collected, or isolated from a subject or patient.

In a preferred embodiment of the invention, said reference value is that of a level, or an activity, or both said level and said activity, of a transcription product of the LIPA gene and/or the CH25H gene, or a translation product of the LIPA gene and/or the CH25H gene, in a sample from a subject not suffering from said neurodegenerative disease.

In preferred embodiments, an alteration in the level and/or activity of a transcription product of the LIPA gene and/or the CH25H gene, or a translation product of the LIPA gene and/or the CH25H gene, in a sample cell, or tissue, or body fluid from said subject relative to a reference value representing a known health status indicates a diagnosis, or prognosis, or increased risk of becoming diseased with a neurodegenerative disease, particulary AD.

The determination of a level of transcription products of the LIPA gene and/or the CH25H gene can be performed in a sample from a subject using a quantitative PCR-analysis with primer combinations to amplify said gene specific sequences from cDNA obtained by reverse transcription of RNA extracted from a sample of a subject. A Northern blot with probes specific for said genes can also be applied. It might further be preferred to measure transcription products by means of chip-based microarray technologies. These techniques are known to those of ordinary skill in the art (see e.g. Sambrook and Russell, Molecular Cloning: A Laboratory Manual, Cold Spring Harbor Laboratory Press, Cold Spring Harbor, N.Y., 2000).

Furthermore, a level and/or activity of a translation product of the LIPA gene (i.e. the lysosomal acid lipase, or acid cholesteryl ester hydrolase)) and/or the CH25H gene (i.e. the cholesterol 25-hydroxylase) can be detected using an immunoassay, an enzyme activity assay, and/or binding assay. These assays can measure the amount of binding between said translation product and an anti-polypeptide antibody by the use of enzymatic, chromodynamic, radioactive, magnetic, or luminescent labels which are attached to either the anti-polypeptide antibody or a secondary antibody which binds the anti-polypeptide antibody. In addition, other high affinity ligands may be used. Immunoassays which can be used include e.g. ELISAs, Western blots and other techniques known to those of ordinary skill in the art (see Harlow and Lane, Antibodies: A Laboratory Manual, Cold Spring Harbor Laboratory Press, Cold Spring Harbor, N.Y., 1999). All these detection techniques may also be employed in the format of microarrays, protein-arrays, antibody arrays, electronic biochip, or protein-chip based technologies. Lysosomal acid lipase enzymatic activity and cholesterol 25-hydroxylase enzymatic activity may be measured by in vitro, cell-based, or in vivo assays. Conveniently, cholesterol 25-hydroxylase enzymatic activity can, for instance, be determined using a hydroxylase activity assay. Likewise, lysosomal acid lipase enzymatic activity can, for instance, be determind using a cholesteryl esterase activity assay.

In a preferred embodiment, the provided methods of diagnosing or prognosticating a neurodegenerative disease in a subject, or determining the propensity or predisposition of a subject to develop such disease, or monitoring a treatment, or evaluating a treatment of a neurodegenerative disease further comprise comparing a level, or an activity, or both said level and said activity, of a transcription product of the LIPA gene and/or the CH25H gene, or a translation product of the LIPA gene and/or the CH25H gene, in a series of samples taken from said subject over a period of time. In another preferred embodiment, said subject receives a treatment prior to one or more sample gatherings. It is a further preferred embodiment to determine said level, or said activity, or both said level and said activity, in said samples before and after said treatment of said subject.

In another aspect, the invention features a kit for diagnosing or prognosticating neurodegenerative diseases, in particular AD, in a subject, or determining the propensity or predisposition of a subject to develop a neurodegenerative disease, in particular AD, said kit comprising:
(a) at least one reagent which is selected from the group consisting of (i) reagents that selectively detect a transcription product of the LIPA gene and/or the CH25H gene, (ii) reagents that selectively detect a translation product of the LIPA gene and/or the CH25H gene, (iii) reagents that selectively detect the presence or absence of a variation in the LIPA gene; and
(b) instruction for diagnosing, or prognosticating a neurodegenerative disease, in particular AD, or determining the propensity or predisposition of a subject to develop such a disease by
   detecting a level, or an activity, or both said level and said activity, of said transcription product and/or said translation product of the LIPA gene and/or the CH25H gene, in a sample from said subject; and/or detecting the presence or absence of a variation in the LIPA gene in a sample from said subject; and
   diagnosing or prognosticating a neurodegenerative disease, in particular AD, or determining the propensity or predisposition of said subject to develop such a disease,
      wherein a varied level, or activity, or both said level and said activity, of said transcription product and/or said translation product compared to a reference value representing a known health status; or a level, or activity, or both said level and said activity, of said transcription product and/or said translation product similar or equal to a reference value representing a known disease status; or the presence of a variation in the LIPA gene, indicates a diagnosis or prognosis of a neurodegenerative disease, in particular AD, or an increased propensity or predisposition of developing such a disease. In a particular embodiment of the instant kit, a diagnosis or prognosis of Alzheimer's disease, or an increased propensity or predisposition to develop Alzheimer's disease in a subject, is indicated by the presence of both variations, the T-variant of SNP rs13500 and the T-variant of SNP rs1131706, in comparison to a subject who does not carry both of said variations, i.e. a subject who carries the C-variant of SNP rs13500 and the A-variant of SNP rs1131706.

It is preferred that the reagents of the kit selectively detect a variation in the 3′-untranslated region of the LIPA gene (single nucleotide polymorphism identification number: rs13500). It is further preferred that the variation is a C to T transition.

It is further preferred that the reagents of the kit selectively detect a variation in the 3′-untranslated region of the LIPA gene (single nucleotide polymorphism identification number: rs1131706). Preferably, said variation is an A to T transversion.

In another preferred embodiment, the kit further comprises reagents that selectively detect the presence or absence of an apolipoprotein E4 allele. The presence of an apolipoprotein E4 allele indicates a diagnosis or prognosis of Alzheimer's disease, or a further increased propensity or predisposition of developing Alzheimer's disease. This embodiment reflects the unexpected synergistic interaction between the genes coding for lysosomal acid lipase and apolipoprotein E4.

In another preferred embodiment, the kit further comprises reagents that selectively detect the presence or absence of a variation in the CYP46 gene. The presence of a variation in the CYP46 gene indicates a diagnosis or prognosis of Alzheimer's disease, or a further increased propensity or predisposition of developing Alzheimer's disease. This embodiment reflects the unexpected synergistic interaction between the genes coding for lysosomal acid lipase (LIPA) and cholesterol 24-hydroxylase (CYP46).

The kit, according to the present invention, may be particularly useful for the identification of individuals that are at risk of developing a neurodegenerative disease, in particular AD. Consequently, the kit, according to the invention, may serve as a means for targeting identified individuals for early preventive measures or therapeutic intervention prior to disease onset, before irreversible damage in the course of the disease has been inflicted. Furthermore, in preferred embodiments, the kit featured in the invention is useful for monitoring a progression of neurodegenerative disease, in particular AD, in a subject. It is further useful in monitoring success or failure of therapeutic treatment for such a disease of said subject.

In another aspect, the invention features a method of treating or preventing a neurodegenerative disease, in particular Alzheimer's disease, in a subject comprising the administration to said subject in a therapeutically or prophylactically effective amount of an agent or agents which directly or indirectly affect a level, or an activity, or both said level and said activity, of (i) of the LIPA gene and/or the CH25H gene, and/or (ii) a transcription product of the LIPA gene and/or the CH25H gene, and/or (iii) a translation product of the LIPA gene and/or the CH25H gene, and/or (iv) a fragment or derivative of (i) to (iii). Said agent may comprise a small molecule, or it may also comprise a peptide, an oligopeptide, or a polypeptide. Said peptide, oligopeptide, or polypeptide may comprise an amino acid sequence of a translation product of the LIPA gene and/or the CH25H gene, or a fragment, or derivative, or a variant thereof. An agent for treating or preventing a neurodegenerative disease, in particular AD, according to the instant invention, may also consist of a nucleotide, an oligonucleotide, or a polynucleotide. Said oligonucleotide or polynucleotide may comprise a nucleotide sequence of the LIPA gene and/or the CH25H gene, either in sense orientation or in antisense orientation.

In preferred embodiments, the method comprises the application of per se known methods of gene therapy and/or antisense nucleic acid technology to administer said agent or agents. In general, gene therapy comprises several approaches: molecular replacement of a mutated gene, addition of a new gene resulting in the synthesis of a therapeutic protein, and modulation of endogenous cellular gene expression by recombinant expression methods or by drugs. Gene-transfer techniques are described in detail (see e.g. Behr, Acc Chem Res 1993, 26:274-278 and Mulligan, Science, 1993, 260: 926-931) and include direct gene-transfer techniques such as mechanical microinjection of DNA into a cell as well as indirect techniques employing biological vectors (like recombinant viruses, especially retroviruses) or model liposomes, or techniques based on transfection with DNA coprecipitation with polycations, cell membrane pertubation by chemical (solvents, detergents, polymers, enzymes) or physical means (mechanic, osmotic, thermic, electric shocks). The postnatal gene transfer into the central nervous system has been described in detail (see e.g. Wolff, Curr Opin Neurobiol 1993, 3:743-748).

In particular, the invention features a method of treating or preventing a neurodegenerative disease by means of antisense nucleic acid therapy, i.e. the down-regulation of an inappropriately expressed or defective gene by the introduction of antisense nucleic acids or derivatives thereof into certain critical cells (see e.g. Gillespie, DN&P 1992, 5:389-395; Agrawal and Akhtar, Trends Biotechnol 1995, 13:197-199; Crooke, Biotechnology 1992, 10:882-6). Apart from hybridization strategies, the application of ribozymes, i.e. RNA molecules that act as enzymes, destroying RNA that carries the message of disease has also been described (see e.g. Barinaga, Science 1993, 262:1512-1514). In preferred embodiments, the subject to be treated is a human, and therapeutic antisense nucleic acids or derivatives thereof are directed against the human LIPA gene and/or the CH25H gene. It is preferred that cells of the central nervous system, preferably the brain, of a subject are treated in such a way. Cell penetration can be performed by known strategies such as coupling of antisense nucleic acids and derivatives thereof to carrier particles, or the above described techniques. Strategies for administering targeted therapeutic oligodeoxynucleotides are known to those of skill in the art (see e.g. Wickstrom, Trends Biotechnol, 1992, 10: 281-287). In some cases, delivery can be performed by mere topical application. Further approaches are directed to intracellular expression of antisense RNA. In this strategy, cells are transformed ex vivo with a recombinant gene that directs the synthesis of an RNA that is complementary to a region of target nucleic acid. Therapeutical use of intracellularly expressed antisense RNA is procedurally similar to gene therapy. A recently developed method of regulating the intracellular expression of genes by the use of double-stranded RNA, known variously as RNA interference (RNAi), can be another effective approach for nucleic acid therapy (Hannon, Nature 2002, 418:244-251).

In further preferred embodiments, the method comprises grafting donor cells into the central nervous system, preferably the brain, of said subject, or donor cells preferably treated so as to minimize or reduce graft rejection, wherein said donor cells are genetically modified by insertion of at least one transgene encoding said agent or agents. Said transgene might be carried by a viral vector, in particular a retroviral vector. The transgene can be inserted into the donor cells by a nonviral physical transfection of DNA encoding a transgene, in particular by microinjection. Insertion of the transgene can also be performed by electroporation, chemically mediated transfection, in particular calcium phosphate transfection or liposomal mediated transfection.

In preferred embodiments, said agent for treating and preventing a neurodegenerative disease, in particular AD, is a therapeutic protein which can be administered to said subject, preferably a human, by a process comprising introducing subject cells into said subject, said subject cells having been treated in vitro to insert a DNA segment encoding said therapeutic protein, said subject cells expressing in vivo in said subject a therapeutically effective amount of said therapeutic protein. Said DNA segment can be inserted into said cells in vitro by a viral vector, in particular a retroviral vector. Said agent, particularly a therapeutic protein, can further be administered to said subject by a process comprising the injection or the systemic administration of a fusion protein, said fusion protein consisting of a fusion of a protein transduction domain with said agent.

Methods of treatment, according to the present invention, comprise the application of therapeutic cloning, transplantation, and stem cell therapy using embryonic stem cells or embryonic germ cells and neuronal adult stem cells, combined with any of the previously described cell- and gene therapeutic methods. Stem cells may be totipotent or pluripotent. They may also be organ-specific. Strategies for repairing diseased and/or damaged brain cells or tissue comprise (i) taking donor cells from an adult tissue. Nuclei of those cells are transplanted into unfertilized egg cells from which the genetic material has been removed. Embryonic stem cells are isolated from the blastocyst stage of the cells which underwent somatic cell nuclear transfer. Use of differentiation factors then leads to a directed development of the stem cells to specialized cell types, preferably neuronal cells (Lanza et al., Nature Medicine 1999, 9: 975-977), or (ii) purifying adult stem cells, isolated from the central nervous system, or from bone marrow (mesenchymal stem cells), for in vitro expansion and subsequent grafting and transplantation, or (iii) directly inducing endogenous neural stem cells to proliferate, migrate, and differentiate into functional neurons (Peterson D A, Curr. Opin. Pharmacol. 2002, 2: 34-42). Adult neural stem cells are of great potential for repairing damaged or diseased brain tissues, as the germinal centers of the adult brain are free of neuronal damage or dysfunction (Colman A, Drug Discovery World 2001, 7: 66-71).

In preferred embodiments, the subject for treatment or prevention, according to the present invention, can be a human, an experimental animal, e.g. a mammal, a mouse, a rat, a fish, an insect, or a worm; a domestic animal, or a non-human primate. The experimental animal can be an animal model for a neurodegenerative disorder, e.g. a transgenic mouse and/or a knock-out mouse with an AD-type neuropathology.

In a further aspect, the invention features a modulator of an activity, or a level, or both said activity and said level of at least one substance which is selected from the group consisting of (i) a gene coding for the LIPA gene and/or the CH25H gene, and/or (ii) a transcription product of the LIPA gene and/or the CH25H gene, and/or (iii) a translation product of the LIPA gene and/or the CH25H gene, and/or (iv) a fragment, or derivative, or variant of (i) to (iii).

In an additional aspect, the invention features a pharmaceutical composition comprising said modulator and preferably a pharmaceutical carrier. Said carrier refers to a diluent, adjuvant, excipient, or vehicle with which the modulator is administered.

In a further aspect, the invention features a modulator of an activity, or a level, or both said activity and said level of at least one substance which is selected from the group consisting of (i) the LIPA gene and/or the CH25H gene, and/or (ii) a transcription product of the LIPA gene and/or the CH25H gene and/or (iii) a translation product of the LIPA gene and/or the CH25H gene, and/or (iv) a fragment, or derivative, or variant of (i) to (iii) for use in a pharmaceutical composition.

In another aspect, the invention provides for the use of a modulator of an activity, or a level, or both said activity and said level of at least one substance which is selected from the group consisting of (i) the LIPA gene and/or the CH25H gene, and/or (ii) a transcription product of the LIPA gene and/or the CH25H gene, and/or (iii) a translation product of the LIPA gene and/or the CH25H gene, and/or (iv) a fragment, or derivative, or variant of (i) to (iii) for a preparation of a medicament for treating or preventing a neurodegenerative disease, in particular AD.

In one aspect, the present invention also provides a kit comprising one or more containers filled with a therapeutically or prophylactically effective amount of said pharmaceutical composition.

In a further aspect, the invention features a recombinant, non-human animal comprising a non-native gene sequence coding for a translation product of the LIPA gene and/or the CH25H gene, or a fragment, or a derivative, or a variant thereof. The generation of said recombinant, non-human animal comprises (i) providing a gene targeting construct containing said gene sequence and a selectable marker sequence, and (ii) introducing said targeting construct into a stem cell of a non-human animal, and (iii) introducing said non-human animal stem cell into a non-human embryo, and (iv) transplanting said embryo into a pseudopregnant non-human animal, and (v) allowing said embryo to develop to term, and (vi) identifying a genetically altered non-human animal whose genome comprises a modification of said gene sequence in both alleles, and (vii) breeding the genetically altered non-human animal of step (vi) to obtain a genetically altered non-human animal whose genome comprises a modification of said endogenous gene, wherein said gene is mis-expressed, or under-expressed, or over-expressed, and wherein said disruption or alteration results in said non-human animal exhibiting a predisposition to developing symptoms of a neurodegenerative disease or related diseases and disorders. Strategies and techniques for the generation and construction of such an animal are known to those of ordinary skill in the art (see e.g. Capecchi, Science, 1989, 244:1288-1292 and Hogan et al., 1994, Manipulating the Mouse Embryo: A Laboratory Manual, Cold Spring Harbor Laboratory Press, Cold Spring Harbor, N.Y.). It is preferred to make use of such a recombinant non-human animal as an animal model for investigating neurodegenerative diseases, in particular Alzheimer's disease.

In another aspect, the invention features an assay for screening for a modulator of neurodegenerative diseases, in particular Alzheimer's disease, or related diseases and disorders of one or more substances selected from the group consisting of (i) the LIPA gene and/or the CH25H gene, and/or (ii) a transcription product of the LIPA gene and/or the CH25H gene, and/or (iii) a translation product of the LIPA gene and/or the CH25H gene, and/or (iv) a fragment, or derivative, or variant of (i) to (iii). This screening method comprises (a) contacting a cell with a test compound, and (b) measuring the level, or the activity, or both the level and the activity of one or more substances recited in (i) to (iv), and (c) measuring the level, or the activity, or both the level and the activity of said substances in a control cell not contacted with said test compound, and (d) comparing the levels of the substance in the cells of step (b) and (c), wherein an alteration in the level and/or activity of said substances in the contacted cells indicates that the test compound is a modulator of said diseases and disorders.

In one further aspect, the invention features a screening assay for a modulator of neurodegenerative diseases, in particular Alzheimer's disease, or related diseases and disorders of one or more substances selected from the group consisting of (i) the LIPA gene and/or the CH25H gene, and/or (ii) a transcription product of the LIPA gene and/or the CH25H gene, and/or (iii) a translation product of the LIPA gene and/or the CH25H gene, and/or (iv) a fragment, or derivative, or variant of (i) to (iii), comprising (a) administering a test compound to a test animal which is predisposed to developing or has already developed symptoms of a neurodegenerative disease or related diseases or disorders, and (b) measuring the level and/or activity of one or more substances recited in (i) to (iv), and (c) measuring the level and/or activity of said substances in a matched control animal which is equally predisposed to developing or has already developed symptoms of a neurodegenerative disease or related disorders and to which animal no such test compound has been administered, and (d) comparing the level and/or activity of the substance in the animals of step (b) and (c), wherein an alteration in the level and/or activity of substances in the test animal indicates that the test compound is a modulator of said diseases and disorders.

In a preferred embodiment, said test animal and/or said control animal is a recombinant, non-human animal which expresses the LIPA gene and/or the CH25H gene, or a fragment, or a derivative, or a variant thereof, under the control of a transcriptional regulatory element which is not the native LIPA gene and/or the CH25H gene transcriptional control regulatory element.

In another embodiment, the present invention provides a method for producing a medicament comprising the steps of (i) identifying a modulator of neurodegenerative diseases by a method of the aforementioned screening assays and (ii) admixing the modulator with a pharmaceutical carrier. However, said modulator may also be identifiable by other types of screening assays.

In another aspect, the present invention provides for a method of testing a compound, preferably an assay for screening a plurality of compounds, for inhibition of binding between a ligand and a LIPA gene product and/or a CH25H gene product, or a fragment or derivative thereof. Said method comprises the steps of (i) adding a liquid suspension of said LIPA gene product and/or CH25H gene product, or a fragment or derivative thereof, to a plurality of containers, and (ii) adding a compound, preferably a plurality of compounds, to be screened for said inhibition to said plurality of containers, and (iii) adding detectable ligand, preferably fluorescently detectable ligand, to said containers, and (iv) incubating the liquid suspension of said LIPA gene product and/or CH25H gene product, or said fragment or derivative thereof, and said compounds, and said detectable ligand, and (v) measuring the amounts of detectable ligand or fluorescence associated with said LIPA gene product and/or CH25H gene product, or with said fragment or derivative thereof, and (vi) determining the degree of inhibition by one or more of said compounds of binding of said ligand to said LIPA gene product and/or CH25H gene product, or said fragment or derivative thereof. Instead of utilizing a fluorescently detectable label, it might in some aspects be preferred to use any other detectable label known to the person skilled in the art, e.g. radioactive label, and detect it accordingly. Said method may be useful for the identification of novel compounds as well as for evaluating compounds which have been improved or otherwise optimized in their ability to inhibit the binding of a ligand to a LIPA gene product and/or a CH25H gene product, or a fragment or derivative thereof. In one further embodiment, the present invention provides a method for producing a medicament comprising the steps of (i) identifying a compound as an inhibitor of binding between a ligand and LIPA gene product and/or CH25H gene product by the aforementioned inhibitory binding assay and (ii) admixing the compound with a pharmaceutical carrier. However, said compound may also be identifiable by other types of screening assays.

In one further aspect, the invention features a method of testing a compound, preferably an assay for screening a plurality of compounds, to determine the degree of binding of said compound or compounds to a LIPA gene product and/or a CH25H gene product, or to a fragment or derivative thereof. Said method comprises the steps of (i) adding a liquid suspension of said LIPA gene product and/or CH25H gene product, or a fragment or derivative thereof, to a plurality of containers, and (ii) adding a detectable compound, preferably a plurality of detectable compounds, in particular fluorescently detectable compounds, to be screened for said binding to said plurality of containers, and (iii) incubating the liquid suspension of said LIPA gene product and/or CH25H gene product, or said fragment or derivative thereof, and said detectable compound, preferably said plurality of detectable compounds, and (iv) measuring the amounts of detectable compound or fluorescence associated with said LIPA gene product and/or CH25H gene product, or with said fragment or derivative thereof, and (v) determining the degree of binding by one or more of said compounds to said LIPA gene product and/or CH25H gene product, or said fragment or derivative thereof. In this type of assay it might be preferred to use a fluorescent label. However, any other type of detectable label might also be employed. Said method may be useful for the identification of novel compounds as well as for evaluating compounds which have been improved or otherwise optimized in their ability to bind to a LIPA gene product and/or a CH25H gene product. In one further embodiment, the present invention provides a method for producing a medicament comprising the steps of (i) identifying a compound as a binder to a LIPA gene product and/or a CH25H gene product by the aforementioned binding assays and (ii) admixing the compound with a pharmaceutical carrier. However, said compound may also be identifiable by other types of screening assays.

In another embodiment, the present invention provides for a medicament obtainable by any of the methods according to the herein claimed screening assays. In one further embodiment, the instant invention provides for a medicament obtained by any of the methods according to the herein claimed screening assays.

The present invention features a protein molecule, said protein molecule being a translation product of the CH25H gene and/or the LIPA gene, or a fragment, or derivative, or variant thereof, for use as a diagnostic target for detecting a neurodegenerative disease, preferably Alzheimer's disease.

The present invention further features a protein molecule, said protein molecule being a translation product of the CH25H gene and/or the LIPA gene, or a fragment, or derivative, or variant thereof, for use as a screening target for reagents, compounds, or substances preventing, or treating, or ameliorating a neurodegenerative disease, preferably Alzheimer's disease.

The present invention features an antibody which is specifically immunoreactive with an immunogen, wherein said immunogen is a translation product of the LIPA gene, or a fragment, or derivative, or variant thereof. Furthermore, the present invention also features an antibody which is specifically immunoreactive with an immunogen, wherein said immunogen is a translation product of the CH25H gene, or a fragment, or derivative, or variant thereof. The immunogen may comprise immunogenic or antigenic epitopes of portions of a translation product of said genes, wherein said immunogenic or antigenic portion of a translation product is a polypeptide, and wherein said polypeptide elicits an antibody response in an animal, and wherein said polypeptide is immunospecifically bound by said antibody. Methods for generating antibodies are well known in the art (see Harlow et al., Antibodies, A Laboratory Manual, Cold Spring Harbor Laboratory Press, Cold Spring Harbor, N.Y.). The term “antibody” encompasses all forms of antibodies known in the art, such as polyclonal, monoclonal, chimeric, recombinatorial, single chain antibodies as well as fragments thereof. Antibodies of the present invention are useful, for instance, in a variety of diagnostic and therapeutic methods involving detecting translation products of the LIPA gene and/or the CH25H gene.

In a preferred embodiment of the present invention, said antibodies can be used for detecting the pathological state of a cell in a sample from a subject, comprising immunocytochemical staining of said cell with said antibody, wherein an altered degree of staining, or an altered staining pattern in said cell compared to a cell representing a known health status indicates a pathological state of said cell. Preferably, the pathological state relates to a neurodegenerative disease, in particular to Alzheimer's disease. Immuno-cytochemical staining of a cell can be carried out by a number of different experimental methods well known in the art. It might be preferred, however, to apply an automated method for the detection of antibody binding, wherein the determination of the degree of staining of a cell, or the determination of the cellular or subcellular staining pattern of a cell, or the topological distribution of an antigen on the cell surface or among organelles and other subcellular structures within the cell, are carried out according to the method described in U.S. Pat. No. 6,150,173.

Other features and advantages of the invention will be apparent from the following description of figures and examples which are illustrative only and not intended to limit the remainder of the disclosure in any way.

Table 1 shows SNP rs13500 genotype and allele distribution in control subjects and Alzheimer's disease patients.

Table 2 shows the unconditional logistic regression analysis (forward and backward) with the diagnosis of Alzheimer's disease as a dependent variable. It also shows the interaction of SNP rs13500 genotype with APOE and CYP46 genotypes and risk for Alzheimer's disease (combined sample).

Table 3 shows the different distribution of the CH25Hz4 haplotype, CH25H*1 T and CH25H*2 A alleles, between AD patients and control subjects in the combined sample. HCS: healthy control subjects; AD: Alzheimer's disease patients.

Table 4 shows brain P-amyloid load differences in the medial temporal lobe between CH25H haplotypes and alleles. Values represent blindedly scored phases of β-amyloid load and are given as median±standard error of the median. Statistical comparisons: H-test (CH25Hχ haplotypes), U-test (CH25H*1 and CH25H*2 alleles).

Table 5 lists CH25H gene expression levels in the hippocampus relative to the frontal cortex in six AD patients (1.32 to 2.69 fold) and three healthy, age-matched control individuals (1.27 to 1.47 fold).

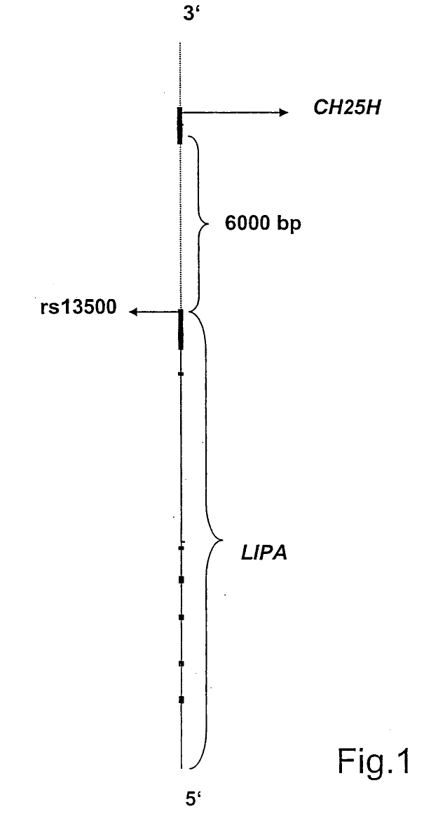

FIG. 1 depicts a schematic representation of the studied genomic region. It demonstrates the close proximity of SNP rs13500 in the LIPA gene in relation to the CH25H gene on human chromosome 10q. SNP information was derived from the database of single nucleotide polymorphisms (dbSNP) established by the National Center for Biotechnology Information (http://www.ncbi.nlm.nih.gov/SNP/index.html).

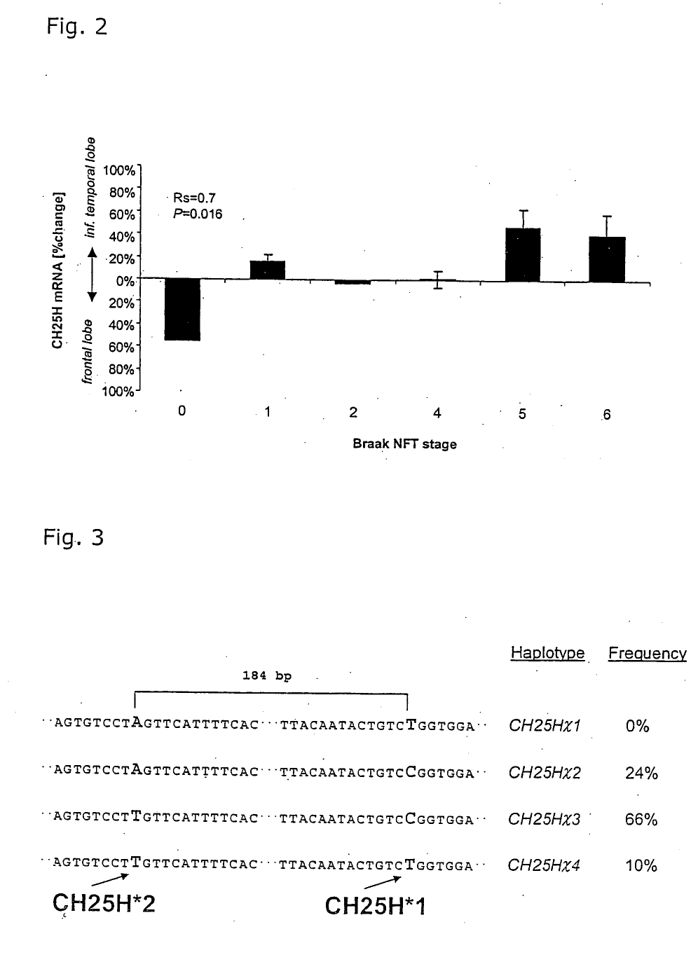

FIG. 2: CH25H mRNA overexpression in vulnerable brain regions in late Braak stages. Expression analysis of CH25H mRNA was done in the inferior temporal lobe and the frontal lobe from brains of 12 aged individuals with Braak stages ranging from 0 to 6. A value of 0% indicates equal CH25H mRNA expression levels in the inferior temporal lobe and the frontal lobe. Bars represent means of expression ratios±SEM. Normalization for housekeeping genes. P=0.016 (Spearman's rank correlation).

FIG. 3: Linkage disequilibrium between SNP CH25H*1 at −6443 bp and SNP CH25H*2 at −6627 bp (relative to the start codon of CH25H). Haplotypes were reconstructed by including individuals homozygous for one or both SNPs. Subjects heterozygous for both SNPs were excluded.

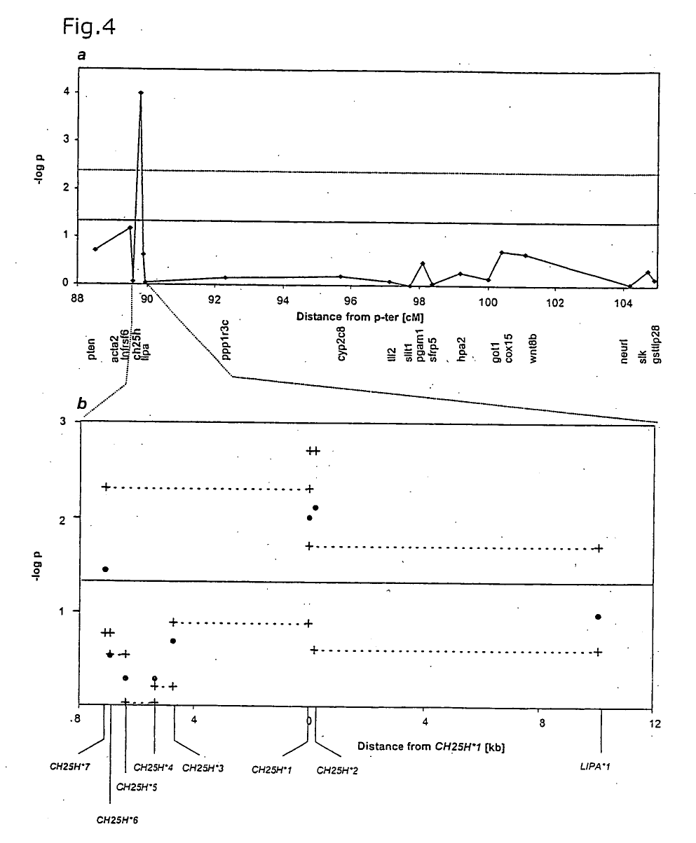

FIG. 4: SNPs in the 5′ UTR of CH25H were significantly associated with AD; (a) Allelic association of SNPs on chromosome 10q with AD. Values on the y-axis represent the negative logarithm of the significance P (χ² test for allelic association). The horizontal continuous line represents the significance level of 0.05, the dotted line represents the significance level after Bonferroni-correction for all analysed SNPs. Distance from p-ter is given in the x-axis in cM according to the NCBI map; (b) Fine-mapping of the CH25H locus at 90 cM. CH25H*1: [T-6443C], corresponds to SNP rs13500; CH25H*2: [A-6627T], corresponds to SNP rs1131706; CH25H*3: [C-1710T]; CH25H*4: [A-1054G]; CH25H*5: [A-44G]; CH25H*6: [T503C]; CH25H*7: [A656G]; LIPA*1: corresponds to SNP rs1556478. SNP positions were calculated relatively to the start codon of CH25H.

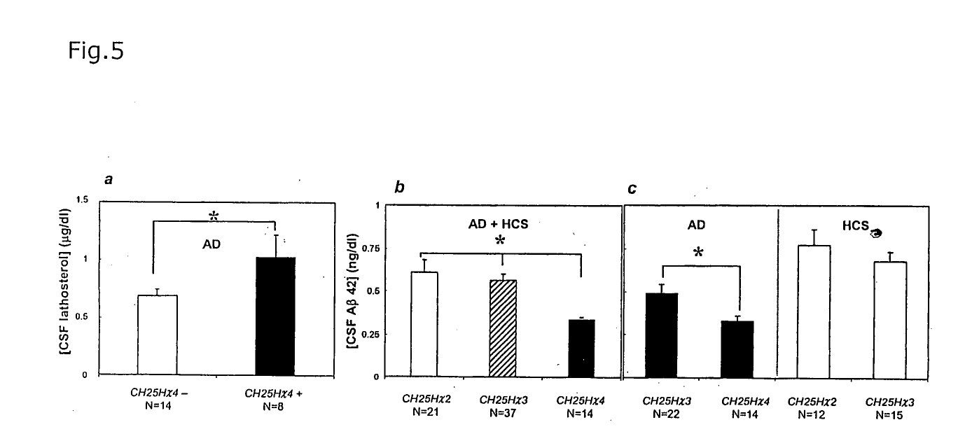

FIG. 5: (a) Higher CSF concentrations of lathosterol in diseased carriers of the CH25Hχ4 haplotype as compared to diseased non-cariers *P=0.046 (Student's t-test); (b) Low CSF concentrations of soluble Aβ₄₂ in CH25Hχ4 carriers, intermediate concentrations in CH25Hχ3 carriers, and high concentrations in CH25Hχ2 carriers *P=0.002 (ANOVA); (c) Significantly lower CSF concentrations of soluble Aβ₄₂ in diseased CH25Hχ4 carriers as compared to diseased CH25Hχ3 carriers *P=0.014 (Student's t-test). The difference between healthy CH25Hχ3 and CH25Hχ2 carriers was not significant; Bars represent means±SEM. AD: AD patients; HCS: healthy control subjects.

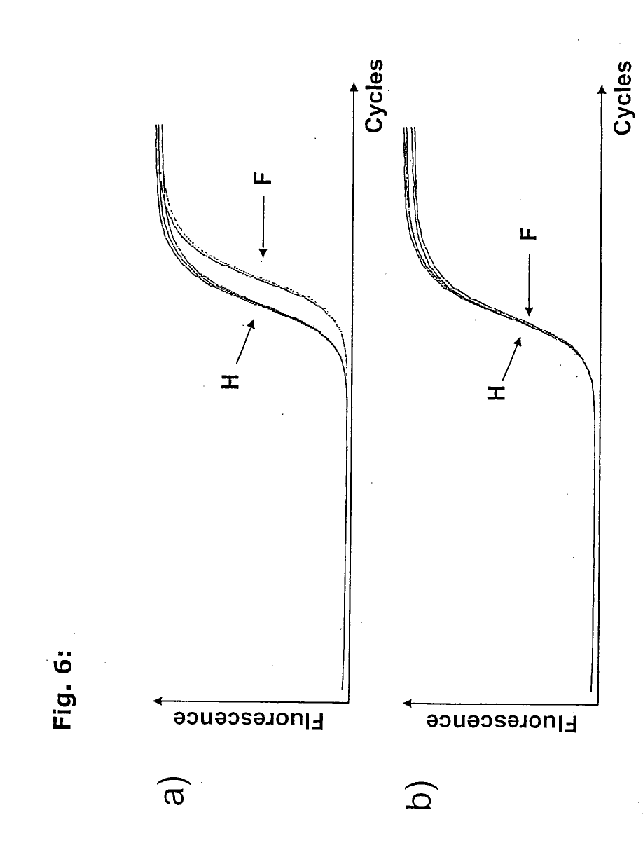

FIG. 6 illustrates the verification of the differential expression of the human CH25H gene in AD brain tissues by quantitative RT-PCR analysis. Quantification of RT-PCR products from RNA samples collected from the frontal cortex (F) and hippocampus (H) of AD patients (FIG. 6a) and of a healthy, age-matched control individuals (FIG. 6b) was performed by the LightCycler rapid thermal cycling technique. The data were normalized to the combined average values of a set of standard genes which showed no significant differences in their gene expression levels. Said set of standard genes consisted of genes for the ribosomal protein S9, the transferrin receptor, GAPDH, cyclophilin B, and beta-actin. The figure depicts the kinetics of amplification by plotting the cycle number against the amount of amplified material as measured by its fluorescence. Note that the amplification kinetics of the CH25H cDNA from both the frontal cortex and the hippocampus of a normal control individual during the exponential phase of the reaction are juxtaposed (FIG. 6b, arrows), whereas in AD (FIG. 6a, arrows), there is a significant separation of the corresponding curves, indicating a differential expression of the CH25H gene in the two analyzed brain regions.

### EXAMPLE 1

To determine whether the single nucleotide polymorphism (SNP) rs13500 in the LIPA gene may be associated with an increased risk for AD, we performed a population-based case-control association study to test for allelic and genotype differences of SNP rs13500 between patients with late-onset AD and non-demented control subjects.

The genotype distribution of SNP rs13500 was that expected under Hardy-Weinberg equilibrium both in patients with AD and in control subjects (p≦0.20). Genotype analysis in the sample revealed a significantly higher frequency of the rs13500*C/T or T/Tgenotype in patients with AD as compared to control subjects (26.4% vs 15.3% respectively, p=0.005) (Table 1). Forward and backward unconditional logistic regression analysis was performed for the simultaneous assessment of the influence of age, rs13500, APOE, and CYP46 genotype on the risk for developing AD (Table 2). Age was included as a continuous variable (1-year intervals). SNP rs13500, APOE and CYP46 genotypes were binomial categorial variables. The analysis of the sample revealed a significant influence of the rs13500, APOE and CYP46 genotypes on the risk for AD (p<0.004; p<0.0001, and p=0.005, respectively). The adjusted odds ratio for the development of AD in APOE*4-carriers was 2.43, the corresponding value for homozygous carriers of the CYP46*T allele was 1.88, the corresponding value for both homozygous or heterozygous carries of the rs13500*T allele was 2.21. (Table 2).

We observed significant interactions between the rs13500 genotype and APOE as well as between the rs13500 genotype and CYP46 (Table 2): Logistic regression analyses predicted a higher impact of these interactions on the risk for AD than each of these genotypes when examined isolated: The interaction term rs13500 and APOE*4 genotype reached an OR of 2.06 with very high significance (95% Cl: 1.45-2.92, p=0.000049). This was also the case for the interaction term CYP46*TT and rs13500 genotype with an OR of 1.81 (95% Cl: 1.30-2.53, p=0.0005).

Subjects and Methods for Genetic Association Studies: Genetic studies were conducted on 422 participants of a Caucasian population. The diagnosis of AD was performed according to the NINCDS-ADRDA criteria based on medical interview, physical examination, neuropsychological testing, brain MRI or CT, as well as blood tests. The mean Mini-Mental State Examination (MMSE) score of the overall patient population (n=193) was 19.7, the mean age was 73.6 years, the mean age-at-onset of AD was 70.5 years. The control group (n=229) comprised cognitively healthy elderly individuals who were either the spouses of AD patients or subjects recruited from the outpatient clinics of the participating institutions. The mean age was 73.7 years and the mean MMSE score was 29.0.

SNP selection and genotyping: Information on polymorphic sites of LIPA and CH25H was derived from the database of single nucleotide polymorphisms (dbSNP) established by the National Center for Biotechnology Information (www.ncbi.nlm.nih.gov/SNP/index.html). Twenty-one SNPs on chromosome 10 were selected for genotyping. Of these 21 potential SNPs, 16 proved to be polymorphic in a sub-sample of 50 participants. Single nucleotide polymorphism rs13500 is located between LIPA and CH25H and predicts a C to T base exchange. SNPs of LIPA and CH25H were genotyped with Masscode-technology according to Kokoris et al. (Mol. Diagn., 5:329-40, 2000) (www.qiagengenomics.com). SNP rs754203 (located 151 bases 5′ to exon 3 of the CYP46 gene) was genotyped by the pyrosequencing method (www.pyrosequencing.com) on a PSQ 96 System. Forward and backward amplification primers for rs754203 were 5′-AAT GCA TGC TAC CAA AAG AG-3′ and 5′-AAT CAT TTG ATT CCC AGG AC-3′, respectively. The backward primer was biotinylated at the 3′ end. Sequencing primer was 5′-GGC AGA GCC TTG CCC-3′. APOE genotyping was performed according to Hixson and Vernier (J Lipid Res, 31:545-8, 1990).

Statistics: Genotype and allelic frequencies between AD patients and controls were compared by Pearson's χ² tests. Forward and backward unconditional logistic regression analyses were done for the simultaneous assessment of the influence of age, gender, APOE, CYP46 and rs13500 genotypes on the risk for developing AD. The estimated haplotype frequencies (EH) program was used to test for LD between SNPs. It computes the maximum-likelihood estimates for the haplotype frequencies assuming no association (H0) and allelic association (H1) and calculates the χ² statistic as the two-fold difference between the log likelihoods (Terwilliger J D, et al., Handbook of Human Genetic Linkage, Baltimore: The Johns Hopkins University Press, 1994, pp.189-198).

### EXAMPLE 2

To determine whether single nucleotide polymorphisms in the genomic region encompassing the CH25H gene and the LIPA gene may be associated with an increased risk for AD, we sequenced the open reading frame and 6.8 kb of the 5′ region of CH25H and identified two synonymous single nucleotide polymorphisms (SNPs), four 3′ SNPs, and six 5′ SNPs. Haplotype analysis revealed three common haplotypes, designated CH25Hχ2, CH25Hχ3 and CH25Hχ4, composed from SNP CH25H*1 at −6443 bp and SNP CH25H*2 at −6627 bp (FIG. 3). The linkage peak on chromosome 10q may result from the combined effect of multiple susceptibility genes. Therefore, we assessed the association between AD and 18 possibly relevant genes within a 20 cM broad region on 10q23-24. Both SNPs and extended haplotypes were analyzed in 353 AD patients and 325 unrelated control subjects from two independent populations. SNP CH25H*1 showed significant allelic association with AD (P=0.0001, FIG. 4a). Fine-mapping of an 18 kb large region around CH25H*1 and subsequent estimated haplotype analysis revealed linkage disequilibrium (LD) between SNPs CH25H*1 and CH25H*2 in a Swiss population (P=0.007) but not in a Mediterranean population (P=0.20). Association mapping of two synonymous SNPs in CH25H, five SNPs in the 5′ region of the gene and three SNPs in the adjacent LIPA gene in the Swiss population revealed significant allelic association of CH25H*1, CH25H*2, and CH25H*7 with AD (FIG. 4b). Haplotype CH25HZ reached the highest significance of association with AD (P=0.0003). Significant allelic and haplotypic association of CH25H*1, CH25H*2 and CH25Hχ with AD was also observed in the combined sample (P=0.0001, P=0.034, P=0.00005, respectively) (Table 3). In the Mediterranean sample, significant association was observed for CH25H*1 and CH25Hχ (P=0.012, P=0.013, respectively), but not for CH25H*2 (P=0.48). An additive interaction between APOE4 and CH25Hχ4 was observed in the combined sample. Compared with individuals lacking the APOE4 allele and the CH25Hχ4 haplotype, the odds ratio (OR) for carriers of both APOE4 and CH25Hχ4 was 5.5 (95% Cl: 2.8-10.9). The OR and 95% Cl was 3.1 (2.1-4.5) for APOE4 carriers and 2.7 (1.5-4.8) for CH25Hχ4 carriers. In addition to CH25Hχ, two additional haplotypes containing CH25H*1 showed significant, yet less pronounced association with AD (FIG. 4b). Seventeen genes within the examined region on 10q failed to show significant allelic association with AD in the study populations.

SNP CH25H*2 is located within the core sequence (CTTG) of the functional binding site for the steroidogenic factor 1 (SF-1) (Quandt et al., Nucleic Acids Res 23:4878-84, 1995; Hu et al., Mol Endocrinol 15:812-8, 2001). SF-1 is involved in the transcriptional regulation of steroid hydroxylases and lipoprotein receptors (Lala et al., Steroids 60:10-4, 1995; Lopez et al., Endocrinology 140:3034-44, 1999). Because allele A of CH25H*2 eliminates the SF-1 binding site, which results in impaired activity of SF-1-dependent regulatory regions, and because CH25H is a potent regulator of cholesterol synthesis, we examined whether the CH25H*2-containing haplotype CH25Hχ is associated with precursors of cholesterol synthesis and found that the concentration of the cholesterol precursor lathosterol in cerebrospinal fluid (CSF) of CH25Hχ4 carriers were significantly higher than in non-carriers (FIG. 5a). These data are compatible with a possible physiologic relevance of this haplotype in transcriptional regulation of CH25H. To explore whether CH25Hχ was pathophysiologically relevant, we examined whether CH25H haplotypes differentially affected β-amyloid plaque pathology. Quantitative neuropathology of brains from 55 elderly subjects (age at death≧60 years) showed that both CH25Hχ4 and CH25Hχ3 were associated with high scores of brain β-amyloid deposition, whereas no β-amyloid deposits were present in CH25Hχ2 carriers (P=0.004, Table 4). In contrast, Braak's NFT staging (Braak and Braak, Acta Neuropathol 82:239-59, 1991) was similar among haplotype groups (P=0.7). The CH25Hχ-related differences in brain β-amyloid deposition were paralleled by low CSF levels of Aβ₄₂ in CH25Hχ4 carriers, intermediate levels in CH25Hχ3 carriers, and high levels in CH25Hχ2 carriers (FIG. 5b,c). By using comprehensive differential display technology (von der Kammer et al., Nucleic Acids Res 27:2211-8, 1999) and real-time quantitative PCR analyses, we observed elevated CH25H gene expression in such vulnerable brain regions as the inferior temporal cortex and the hippocampus in AD patients (FIG. 2).

Subjects and Methods for Genetic Association Studies: Genetic studies were conducted on 2 independent populations: a Swiss sample (342 participants) and a Mediterranean sample (336 participants from Greece and Italy). The diagnosis of AD was performed according to the NINCDS-ADRDA criteria based on medical interview, physical examination, neuropsychological testing, brain MRI or CT, as well as blood tests. The mean Mini-Mental State Examination (MMSE) score of the patient population (n=353) was 20.7, the mean age was 72.9 years, the mean age of onset of AD was 69.5 years. There were 215 (60.9%) female participants in the patient group. The control group (n=325) comprised cognitively healthy elderly individuals who were either the spouses of AD patients or subjects recruited from the outpatient clinics of the participating institutions. The mean age was 71.1 years and the mean MMSE score was 29.0. There were 172 (52.9%) female participants in this group.

Neuropathological Methods: Neuropathological examinations were performed in the brains of 55 elderly individuals (mean age of death: 71.3 years, range 60-91 years, 23 females) devoid of significant neuropathological abnormalities and without signs of dementia, as measured by the Clinical Dementia Rating (CDR) scale (Hughes et al., Br J Psychiatry 140:566-72, 1982). The evolutionary phases (0-4) of β-amyloidosis in the medial temporal lobe of these subjects were determined as described by Thal et al. (J Neuropathol Exp Neurol 59:733-48, 2000; Neurology 58:1791-800, 2002). Neurofibrillary tangle (NFT) staging (0-6) was performed according to Braak and Braak (supra). For genotype determination, DNA was extracted from cerebellar fresh frozen tissue samples following standard protocols.

Brain tissue dissection from patients with AD: Brain tissues from AD patients and age-matched control subjects were collected within 5 hours post-mortem and immediately frozen on dry ice. Sample sections from each tissue were fixed in paraformaldehyde for histopathological confirmation of the diagnosis. Brain areas for differential expression analysis were identified and stored at −80° C. until RNA extractions were performed.

Isolation of total mRNA: Total RNA was extracted from post-mortem brain tissue by using the RNeasy kit (Qiagen) according to the manufacturer's protocol. The accurate RNA concentration and the RNA quality were determined with the DNA LabChip system using the Agilent 2100 Bioanalyzer (Agilent Technologies). For additional quality testing of the prepared RNA, i.e. exclusion of partial degradation and testing for DNA contamination, specifically designed intronic GAPDH oligonucleotides and genomic DNA as reference control were used to generate a melting curve with the LightCycler technology as described in the manufacturer's protocol (Roche).

Differential mRNA expression by quantitative RT-PCR: In order to compare RNA populations from carefully selected post-mortem brain tissues (hippocampus, frontal and inferior temporal cortex) and to analyze the differential expression of the gene coding for CH25H, qPCR using the LightCycler technology (Roche) was employed. This technique features rapid thermal cyling for the polymerase chain reaction as well as real-time measurement of fluorescent signals during amplification and therefore allows for highly accurate quantification of RT-PCR products by using a kinetic, rather than an endpoint readout. The ratio of CH25H cDNA from the hippocampus or temporal cortex and frontal cortex was determined (relative quantification).

First, a standard curve was generated to determine the efficiency of the PCR with specific primers for the gene coding for CH25H:

5′-GGTCAACATCTGGCTTTCCG -3′
and
5′-CACCAGTCTGTGAGTGGACCAA -3′.

PCR amplification (95° C. and 1 sec, 56° C. and 5 sec, and 72° C. and 5 sec) was performed in a volume of 20 μl containing LightCycler-FastStart DNA Master SYBR Green I mix (contains FastStart Taq DNA polymerase, reaction buffer, dNTP mix with dUTP instead of dTTP, SYBR Green I dye, and 1 mM MgCl₂; Roche), 0.5 μM primers, 2 μl of a cDNA dilution series (final concentration of 40, 20, 10, 5, 1 and 0.5 ng human total brain cDNA; Clontech) and, depending on the primers used, additional 3 mM MgCl₂. Melting curve analysis revealed a single peak at approximately 84.3° C. with no visible primer dimers. Quality and size of the PCR product were determined with the DNA LabChip system (Agilent 2100 Bioanalyzer, Agilent Technologies). A single peak at the expected size of 70 bp for the CH25H gene was observed in the electropherogram of the sample.

In an analogous manner, the PCR protocol was applied to determine the PCR efficiency of a set of reference genes which were selected as a reference standard for quantification. In the present invention, the mean value of five such reference genes was determined: (1) cyclophilin B, using the specific primers 5′-ACTGAAGCACTACGGGCCTG-3′ and 5′-AGCCGTTGGTGTCTT TGCC-3′ except for MgCl₂ (an additional 1 mM was added instead of 3 mM). Melting curve analysis revealed a single peak at approximately 87° C. with no visible primer dimers. Agarose gel analysis of the PCR product showed one single band of the expected size (62 bp). (2) Ribosomal protein S9 (RPS9), using the specific primers 5′-GGTCAAATTTACCCTGGCCA-3′ and 5′- TCTCATCAAGCGTCAGCAGTTC-3′ (exception: additional 1 mM MgCl₂ was added instead of 3 mM). Melting curve analysis revealed a single peak at approximately 85° C. with no visible primer dimers. Agarose gel analysis of the PCR product showed one single band with the expected size (62 bp). (3) beta-actin, using the specific primers 5′-TGGAACGGTGAAGGTGACA-3′ and 5′-GGCAAGGGACTTCCTGTAA-3′ . Melting curve analysis revealed a single peak at approximately 87° C. with no visible primer dimers. Agarose gel analysis of the PCR product showed one single band with the expected size (142 bp). (4) GAPDH, using the specific primers 5′-CGTCATGGGTGTGAACCATG-3′ and 5′-GCTAAGCAGTTGGTGGTGCAG-3′. Melting curve analysis revealed a single peak at approximately 83° C. with no visible primer dimers. Agarose gel analysis of the PCR product showed one single band with the expected size (81 bp). (5) Transferrin receptor TRR, using the specific primers 5′-GTCGCTGGTCAGTTCGTGATT-3′ and 5′-AGCAGTTGGCTGTTGTACCTCTC-3′. Melting curve analysis revealed a single peak at approximately 83° C. with no visible primer dimers. Agarose gel analysis of the PCR product showed one single band with the expected size (80 bp).

For calculation of the values, first the logarithm of the cDNA concentration was plotted against the threshold cycle number Cₜ for the gene coding for CH25H and the five reference standard genes. The slopes and the intercepts of the standard curves (i.e. linear regressions) were calculated for all genes. In a second step, cDNAs from frontal cortex and hippocampus were analyzed in parallel and normalized to cyclophilin B. The Cₜ values were measured and converted to ng total brain cDNA using the corresponding standard curves:
10\hat{ }((Cₜ value−intercept)/slope)[ng total brain cDNA]

The values for frontal cortex and hippocampus CH25H cDNAs were normalized to cyclophilin B and the ratio was calculated according to formula:

$$Ratio = \frac{CH25H hippocampus [ ng ] / cyclophilin B hippocampus [ ng ]}{CH25H frontal [ ng ] / cyclophilin B frontal [ ng ]}$$

In a third step, the set of reference standard genes was analyzed in parallel to determine the mean average value of the hippocampus to frontal cortex ratios of expression levels of the reference standard genes for each individual brain sample. As cyclophilin B was analyzed in step 2 and step 3, and the ratio from one gene to another gene remained constant in different runs, it was possible to normalize the values for CH25H to the mean average value of the set of reference standard genes instead of normalizing to one single gene alone. The calculation was performed by dividing the ratio shown above by the deviation of cyclophilin B from the mean value of all housekeeping genes. The results of one such quantitative RT-PCR analysis for the CH25H gene are shown in FIG. 6.

Cerebrospinal fluid analysis: CSF was obtained by lumbar puncture in a subset of the participants of the genetic studies in Zurich. Forty-five AD patients (mean age: 70.1 years) and 27 healthy elderly subjects (mean age: 65.4 years) were included. For CSF A(142 analysis, we used a sandwich ELISA (INNOTEST β-Amyloid 1-42, Innogenetics) with mAb 21F12, specific for the free C-terminal end of Aβ₄₂ (peptide sequence Aβ 33-42), as capturing antibody and the mAb 3D6, specific for the N-terminal end of Aβ₄₂ (peptide sequence Aβ 1-5), as detector. CSF lathosterol was measured by means of combined gas chromatography/mass spectrometry (Dzeletovic et al., Anal Biochem 225:73-80, 1995).

SNP Selection and Genotyping: Information on polymorphic sites of 10q23-24 was derived from the database of single nucleotide polymorphisms (dbSNP) established by the National Center for Biotechnology Information (http://www.ncbi.nlm.nih.gov/SNP/index.html). The following genes were selected: PTEN (Phosphatase and tensin homolog), ACTA2 (Alpha 2 actin), TNFRSF6 (Tumor necrosis factor receptor superfamily, member 6), CH25H (Cholesterol 25-hydroxylase), LIPA (Lipase A), PPP1R3C (Protein phosphatase 1, regulatory subunit 3C), CYP2C8 (cytochrome P450, subfamily IIC (mephenytoin 4-hydroxylase), polypeptide 8), TLL2 (Tolloid-like 2), SLIT1 (Slit homolog 1), PGAMI (Phosphoglycerate mutase 1), SFRP5 (Secreted frizzled-related protein 5), HPA2 (Heparanase 2), GOT1 (Glutamic-oxaloacetic transaminase 1), COX15 (COX15 homolog), WNT8B (Wingless-type MMTV integration site family), NEURL (Neuralized-like), SLK (Ste20-related serine/threonine kinase), and GSTTLp28 (Glutathione-S-transferase like). SNP-identification in the ORF, the 5′ and the 3′ region of CH25H was done by double-stranded sequencing of 40 chromosomes on an ABI PRISM 310 Genetic Analyzer. The Masscode system was used for SNP genotyping (Kokoris et al., Mol Diagn 5:329-40, 2000).

Statistics: Genotype and allelic frequencies between AD patients and controls were compared by Fisher's exact tests. Forward and backward unconditional logistic regression analyses were done for the simultaneous assessment of the influence of age, gender, APOE and CH25H genotypes on the risk for developing AD. The estimate haplotype frequencies program was used to test for LD between SNPs and for significance of haplotype distribution between AD cases and controls (Terwilliger et al., supra). Phases of β-amyloidosis between groups were compared with the U-test by Wilcoxon, Mann and Whitney. The significance of correlation between CH25H mRNA expression levels and NFT stages was assessed by the Spearman's rank correlation coefficient. For the comparison of CSF Aβ₄₂ and lathosterol levels, t-tests were used.

TABLE 1
SNP rs13500 genotype distribution in control subjects and AD patients

| | Control subjects | AD patients |
|---|---|---|
| | (n = 229) | (n = 193) |
| rs13500 | | |
| genotype | | |
| C/C | 194 (84.7%) | 142 (73.6%) |
| C/T | 35 (15.3%) | 48 (24.9%) |
| T/T | 0 (0%) | 3 (1.6%) |

| | |
|---|---|
| Statistics | Pearson's χ² = 10.1 |
| | p = 0.006 |

| | | |
|---|---|---|
| rs13500 | | |
| genotype | | |
| C/C | 194 (84.7%) | 142 (73.6%) |
| C/T or T/T | 35 (15.3%) | 51 (26.4%) |

| | |
|---|---|
| Statistics | Pearson's χ² = 8.0 |
| | p = 0.005 |

TABLE 2
Unconditional logistic regression analysis (forward and
backward) with the diagnosis of AD as dependent variable

| | | Adjusted | |
|---|---|---|---|
| Independent variable | Significance (p) | Odds ratio | 95% Cl |
| Age (1-year intervals) | 0.000006 | 1.06 | |
| APOE*4 allele | 0.0001 | 2.43 | 1.55-3.79 |
| CYP46*TT genotype | 0.005 | 1.88 | 1.21-2.92 |
| rs13500 genotype | 0.004 | 2.21 | 1.29-3.77 |
| Age | 0.000005 | 1.06 | 1.04-1.09 |
| Interaction: | 0.000049 | 2.06 | 1.45-2.92 |
| (APOE*4 allele × rs13500) | | | |
| Interaction: | 0.0005 | 1.81 | 1.30-2.53 |
| (CYP46*TT × rs13500) | | | |

TABLE 3

| | HCS (n = 325) | AD (n = 353) | P |
|---|---|---|---|
| CH25Hχ4 haplotype | 10.8% | 22.4% | 0.00005 |
| CH25H*1 T allele | 6.5% | 12.6% | 0.0001 |
| CH25H*2 A allele | 13.7% | 9.9% | 0.034 |

HCS: healthy control subjects;
AD: AD patients

TABLE 4

| | n | brain β-amyloid load | P |
|---|---|---|---|
| CH25Hχ haplotype | | | |
| χ4 | 6 | 1.5 ± 0.9 | |
| χ3 | 33 | 1.0 ± 0.6 | 0.004 |
| χ2 | 16 | 0.0 ± 0.0 | |
| CH25H*B 1 T allele | | | |
| T+ | 6 | 1.5 ± 0.9 | 0.302 |
| T− | 49 | 0.0 ± 0.3 | |
| CH25H*2 A allele | | | |
| A+ | 16 | 0.0 ± 0.0 | 0.001 |
| A− | 39 | 1.0 ± 0.6 | |

TABLE 5

| | Δ (fold) |
|---|---|
| sample | (hippocampus/frontal cortex) |
| patient 1 | 2.56 |
| patient 2 | 2.01 |
| patient 3 | 2.69 |
| patient 4 | 1.79 |
| patient 5 | 2.10 |
| patient 6 | 1.32 |
| control 1 | 1.27 |
| control 2 | 1.40 |
| control 3 | 1.47 |

1-38. (canceled)

39. A method for diagnosing or prognosticating Alzheimer's disease in a subject, or determining the propensity or predisposition of a subject to develop Alzheimer's disease, which comprises detecting in a sample obtained from said subject the presence or absence of a variation in the LIPA gene, wherein the presence of the variation in the LIPA gene in the sample from said subject indicates a diagnosis or prognosis of Alzheimer's disease, or an increased propensity or predisposition to develop Alzheimer's disease as compared to a subject who does not carry the variation in said gene.

40. The method of claim 39, wherein said variation in the LIPA gene is a single nucleotide polymorphism in the 3′-untranslated region (single nucleotide polymorphism identification number: rs13500).

41. The method of claim 40, wherein said variation is a C to T transition.

42. The method of claim 39, wherein said variation in the LIPA gene is a single nucleotide polymorphism in the 3′-untranslated region (single nucleotide polymorphism identification number: rs1131706).

43. The method of claim 42, wherein said variation is an A to T transversion.

44. The method of claim 39, wherein the variation is a C to T transition variation, an A to T transversion variation, or both a C to T transition variation and an A to T transversion variation.

45. The method of claim 44, further comprising detecting in the sample from said subject the presence of an apolipoprotein E4 allele.

46. The method of claim 45, wherein the presence of the apolipoprotein E4 allele and the presence of one or both of the variations in the LIPA gene in said subject indicates a diagnosis or prognosis of Alzheimer's disease, or a further increased propensity or predisposition to develop Alzheimer's disease.

47. The method of claim 44, further comprising detecting in the sample from said subject the presence of a variation in the CYP46 gene.

48. The method of claim 47, wherein said variation in the CYP46 gene is a single nucleodide polymorphism at a position 151 bp 5′ of exon 3 (single nucleotide polymorphism identification number: rs754203).

49. The method of claim 48, wherein said variation is a C to T transition.

50. The method of claim 47, wherein the presence of the variation in the CYP46 gene and the presence of one or both of the variations in the LIPA gene in said subject indicates a diagnosis or prognosis of Alzheimer's disease, or a further increased propensity or predisposition to develop Alzheimer's disease.

51. A method for diagnosing or prognosticating a neurodegenerative disease in a subject, or determining the propensity or predisposition of a subject to develop said disease, comprising determining in a sample from said subject a level and/or an activity of at least one substance which is selected from the group consisting of
(i) a transcription product of the CH25H gene and/or the LIPA gene,
(ii) a translation product of the CH25H gene and/or the LIPA gene,
(iii) a fragment, or derivative, or variant of said transcription or translation product,
and comparing said level and/or said activity to a reference value representing a known disease or health status, thereby diagnosing or prognosticating said neurodgenerative disease in said subject, or determining the propensity or predisposition of said subject to develop said neurodegenerative disease.

52. The method according to claim 51 wherein said neurodegenerative disease is Alzheimer's disease.

53. A kit for diagnosing or prognosticating a neurodegenerative disease, in particular Alzheimer's disease, in a subject, or determining the propensity or predisposition of a subject to develop such a disease by:
(i) detecting in a sample obtained from a subject a varied level and/or an activity of a transcription product and/or of a translation product of the CH25H gene and/or the LIPA gene compared to a reference value; and/or
(ii) detecting in a sample obtained from said subject the presence or absence of a variation in the LIPA gene,
and said kit comprising:
at least one reagent which is selected from the group consisting of (i) reagents that selectively detect a transcription product of the CH25H gene and/or the LIPA gene, (ii) reagents that selectively detect a translation product of the CH25H gene and/or the LIPA gene, (iii) reagents that selectively detect the presence or absence of a variation in the LIPA gene.

54. The kit according to claim 53, wherein said variation in the LIPA gene is a single nucleotide polymorphism in the 3′-untranslated region (single nucleotide polymorphism identification number: rs13500).

55. The kit according to claim 54, wherein the variation is a C to T transition.

56. The kit of claim 53, wherein said variation in the LIPA gene is a single nucleodide polymorphism in the 3′-untranslated region (single nucleotide polymorphism identification number: rs1131706).

57. The kit of claim 56, wherein said variation is an A to T transversion.

58. The kit of claim 53, wherein the variation is a C to T transition variation, an A to T transversion variation, or both a C to T transition variation and an A to T transversion variation.

59. The kit according to claim 58,further comprising reagents that selectively detect the presence or absence of an apolipoprotein E4 allele.

60. The kit according to claim 59, wherein the presence of the apolipoprotein E4 allele and the presence or absence of a variation in the LIPA gene indicates a diagnosis or prognosis of a neurodegenerative disease, in particular Alzheimer's disease, or a further increased propensity or predisposition of developing such a disease.

61. The kit according to claim 53, further comprising reagents that selectively detect the presence or absence of a variation in the CYP46 gene.

62. The kit of claim 61, wherein said variation in the CYP46 gene is a single nucleotide polymorphism at a position 151 bp 5′ of exon 3 (single nucleotide polymorphism identification number: rs754203).

63. The kit of claim 62, wherein said variation is a C to T transition.

64. The kit according to claim 61, wherein the presence of the variation in the CYP46 gene and the presence or absence of the variation in the LIPA gene in said subject indicates a diagnosis or prognosis of a neurodegenerative disease, in particular Alzheimer's disease, or a further increased propensity or predisposition to develop such a disease.

65. A method of treating or preventing a neurodegenerative disease, in particular Alzheimer's disease, in a subject comprising administering to said subject in a therapeutically or prophylactically effective amount an agent or agents which directly or indirectly affect a level and/or activity of at least one substance which is selected from the group consisting of the CH25H gene and/or the LIPA gene, or a transcription product of the CH25H gene and/or the LIPA gene, or a translation product of the CH25H gene and/or the LIPA gene, in a sample from said subject.

66. A modulator of an activity and/or of a level of at least one substance which is selected from the group consisting of (i) the CH25H gene and/or the LIPA gene, and/or (ii) a transcription product of the CH25H gene and/or the LIPA gene, and/or (iii) a translation product of the CH25H gene and/or the LIPA gene, and/or (iv) a fragment, or derivative, or variant of (i) to (iii).

67. A recombinant, non-human animal comprising a non-native gene sequence coding for a translation product of the CH25H gene and/or the LIPA gene, or a fragment, or a derivative, or a variant thereof, said animal being obtainable by:
(i) providing a gene targeting construct comprising said gene sequence and a selectable marker sequence, and
(ii) introducing said targeting construct into a stem cell of a non-human animal, and
(iii) introducing said non-human animal stem cell into a non-human embryo, and
(iv) transplanting said embryo into a pseudopregnant non-human animal, and
(v) allowing said embryo to develop to term, and
(vi) identifying a genetically altered non-human animal whose genome comprises a modification of said gene sequence in both alleles, and
(vii) breeding the genetically altered non-human animal of step (vi) to obtain a genetically altered non-human animal whose genome comprises a modification of said endogenous gene, wherein said disruption results in said non-human animal exhibiting a predisposition to developing symptoms of a neurodegenerative disease or related diseases or disorders.

68. Use of the recombinant, non-human animal according to claim 67 as an animal model for investigating neurodegenerative diseases, in particular Alzheimer's disease.

69. An assay for screening for a modulator of neurodegenerative diseases, in particular Alzheimer's disease, or related diseases or disorders of one or more substances selected from the group consisting of
(i) the Ch25H gene and/or the LIPA gene, and/or
(ii) a transcription product of the CH25H gene and/or the LIPA gene, and/or
(iii) a translation product of the CH25H gene and/or the LIPA gene, and/or
(iv) a fragment, or derivative, or variant of (i) to (iii), said method comprising:
(a) contacting a cell with a test compound;
(b) measuring the activity and/or level of one or more substances recited in (i) to (iv);
(c) measuring the activity and/or level of one or more substances recited in (i) to (iv) in a control cell not contacted with said test compound; and
(d) comparing the levels and/or activities of the substance in the cells of step (b) and (c), wherein an alteration in the activity and/or level of substances in the contacted cells indicates that the test compound is a modulator of said diseases or disorders.

70. A method of screening for a modulator of neurodegenerative diseases, in particular Alzheimer's disease, or related diseases or disorders of one or more substances selected from the group consisting of
(i) the CH25H gene and/or the LIPA gene, and/or
(ii) a transcription product of the CH25H gene and/or the LIPA gene, and/or
(iii) a translation product of the CH25H gene and/or the LIPA gene, and/or
(iv) a fragment, or derivative, or a variant of (i) to (iii), said method comprising:
(a) administering a test compound to a test animal which is predisposed to developing or has already developed symptoms of a neurodegenerative disease or related diseases or disorders in respect of the substances recited in (i) to (iv);
(b) measuring the activity and/or level of one or more substances recited in (i) to (iv);
(c) measuring the activity and/or level of one or more substances recited in (i) or (iv) in a matched control animal which is predisposed to developing or has already developed symptoms of a neurodegenerative disease or related diseases or disorders in respect to the substances recited in (i) to (iv) and to which animal no such test compound has been administered;
(d) comparing the activity and/or level of the substance in the animals of step (b) and (c), wherein an alteration in the activity and/or level of substances in the test animal indicates that the test compound is a modulator of said diseases or disorders.

71. The method according to claim 70 wherein said test animal and/or said control animal is a recombinant animal which expresses a CH25H gene product and/or a LIPA gene product, or a fragment, or a derivative, or a variant thereof, under the control of a transcriptional control element which is not the native CH25H gene and/or LIPA gene transcriptional control element.

72. A method of testing a compound, preferably of screening a plurality of compounds, for inhibition of binding between a ligand and a CH25H gene product and/or a LIPA gene product, or a fragment, or derivative, or variant thereof, said method comprising the steps of:
(i) adding a liquid suspension of said CH25H gene product and/or LIPA gene product, or a fragment or derivative thereof, to a plurality of containers;
(ii) adding a compound, preferably a plurality of compounds, to be screened for said inhibition of binding to said plurality of containers;
(iii) adding a detectable ligand, in particular a fluorescently detectable ligand, to said containers;
(iv) incubating the liquid supension of said CH25H gene product and/or LIPA gene product, or said fragment, or derivative, or variant thereof, and said compound, preferably said plurality of compounds, and said ligand;
(v) measuring amounts of detectable ligand or fluorescence associated with said CH25H gene product and/or LIPA gene product, or with said fragment, or derivative, or variant thereof; and
(vi) determining the degree of inhibition by one or more of said compounds of binding of said ligand to said CH25H gene product and/or LIPA gene product, or said fragment, or derivative, or variant thereof.

73. A method of testing a compound, preferably of screening a plurality of compounds, to determine the degree of binding of said compound or compounds to a CH25H gene product and/or LIPA gene product, or to a fragment, or derivative, or variant thereof, said method comprising the steps of:
(i) adding a liquid suspension of said CH25H gene product and/or LIPA gene product, or a fragment, or derivative, or variant thereof, to a plurality of containers;
(ii) adding a detectable compound, preferably a plurality of detectable compounds, in particular fluorescently detectable compounds, to be screened for said binding to said plurality of containers;
(iii) incubating the liquid suspension of said CH25H gene product and/or LIPA gene product, or said fragment, or derivative, or variant thereof, and said compound, preferably said plurality of compounds;
(iv) measuring amounts of detectable compound or fluorescence associated with said CH25H gene product and/or LIPA gene product, or with said fragment, or derivative, or variant thereof; and
(v) determining the degree of binding by one or more of said compounds to said CH25H gene product and/or LIPA gene product, or said fragment, or derivative, or variant thereof.

74. An antibody specifically immunoreactive with an immunogen, wherein said immunogen is a translation product of the CH25H gene, or a fragment, or derivative, or variant thereof.

75. An antibody specifically immunoreactive with an immunogen, wherein said immunogen is a translation product of the LIPA gene, or a fragment, or derivative, or variant thereof.

76. Use of an antibody according to claim 74 for detecting the pathological state of a cell in a sample from a subject, comprising immunocytochemical staining of said cell with said antibody, wherein an altered degree of staining, or an altered staining pattern in said cell compared to a cell representing a known health status indicates a pathological state of said cell.

77. The kit of claim 53, wherein said variation in the LIPA gene is both a single C to T transition polymorphism in the 3′-untranslated region (single nucleotide polymorphism identification number: rs13500) and a single A to T transversion polymorphism in the 3′-untranslated region (single nucleotide polymorphism identification number: rs1131706), wherein the presence of both the C to T transition variation and the A to T transversion variation in said subject indicates a diagnosis or prognosis of a neurodegenerative disease, in particular Alzheimer's disease, or an increased propensity or predisposition to develop such a disease, as compared to a subject who does not carry both variations.

78. The method of claim 39, wherein said variation in the LIPA gene is both a single C to T transition polymorphism in the 3′-untranslated region (single nucleotide polymorphism identification number: rs13500) and a single A to T transversion polymorphism in the 3′-untranslated region (single nucleotide polymorphism identification number: rs1131706), wherein the presence of both the C to T transition variation and the A to T transversion variation in said subject indicates a diagnosis or prognosis of a neurodegenerative disease, in particular Alzheimer's disease, or an increased propensity or predisposition to develop such a disease, as compared to a subject who does not carry both variations.
